SEQUENCE LISTING (40 sequences)

SEQ ID NO: 1 - DNA, 22 bp, Artificial Sequence
  Primer
agaccaagatggtcagaggatc

SEQ ID NO: 2 - DNA, 22 bp, Artificial Sequence
  Primer
actggaacacagtaaagcaagc

SEQ ID NO: 3 - DNA, 21 bp, Artificial Sequence
  Primer
aaattaaccggaatgggtttg

SEQ ID NO: 4 - DNA, 21 bp, Artificial Sequence
  Primer
atgactcagattgatgcctgg

SEQ ID NO: 5 - DNA, 21 bp, Artificial Sequence
  Primer
ggctcctcttaacccaaaggc

SEQ ID NO: 6 - DNA, 22 bp, Artificial Sequence
  Primer
cacaccatcaccagaatccagc

SEQ ID NO: 7 - DNA, 20 bp, Artificial Sequence
  Primer
ttgcaaccaggcaccttgaa

SEQ ID NO: 8 - DNA, 20 bp, Artificial Sequence
  Primer
caaatcggcgggcattatgt

SEQ ID NO: 9 - DNA, 22 bp, Artificial Sequence
  Primer
tggtgctggacatttcagtcgg

SEQ ID NO: 10 - DNA, 24 bp, Artificial Sequence
  Primer
caagagcttgcacttccatcatag

SEQ ID NO: 11 - DNA, 20 bp, Artificial Sequence
  Primer
cgaggaaggatctcttgcag

SEQ ID NO: 12 - DNA, 20 bp, Artificial Sequence
  Primer
gcactagtgaaccccagagg

SEQ ID NO: 13 - DNA, 24 bp, Artificial Sequence
  Primer
ctcaaaatcccacgcttcttgtgg

SEQ ID NO: 14 - DNA, 24 bp, Artificial Sequence
  Primer
cacgtctactacctttggtttccc

SEQ ID NO: 15 - DNA, 20 bp, Artificial Sequence
  Primer
ctcgagatggacgaagaagg

SEQ ID NO: 16 - DNA, 20 bp, Artificial Sequence
  Primer
cgacgcagagtaatcgaaca

SEQ ID NO: 17 - DNA, 53 bp, Artificial Sequence
  Primer
ggggacaagtttgtacaaaaaagcaggcttcatatgaaaacctaattcctctg

SEQ ID NO: 18 - DNA, 52 bp, Artificial Sequence
  Primer
ggggaccactttgtacaagaaagctgggtcggtcagacaatcgtaagctggt

SEQ ID NO: 19 - DNA, 25 bp, Artificial Sequence
  Primer
atggataatgaacctaggagatatg

SEQ ID NO: 20 - DNA, 28 bp, Artificial Sequence
  Primer
ctcctaggttcattatccatgcccattc

SEQ ID NO: 21 - DNA, 30 bp, Artificial Sequence
  Primer
aggagatatgggcccaactatacacacttg

SEQ ID NO: 22 - DNA, 25 bp, Artificial Sequence
  Primer
tagttgggcccatatctcctaggtt

SEQ ID NO: 23 - protein, 7 aa, Arabidopsis thaliana
PFKKMKV

SEQ ID NO: 24 - DNA, 53 bp, Artificial Sequence
  Primer
ggggacaagtttgtacaaaaaagcaggcttcatgggaggaccagcttacgatt

SEQ ID NO: 25 - DNA, 52 bp, Artificial Sequence
  Primer
ggggaccactttgtacaagaaagctgggtcttacttgagcctctcctttcta

SEQ ID NO: 26 - DNA, 53 bp, Artificial Sequence
  Primer
ggggacaagtttgtacaaaaaagcaggcttcatgtcaggatgggacgaaggag

SEQ ID NO: 27 - DNA, 53 bp, Artificial Sequence
  Primer
ggggaccactttgtacaagaaagctgggtcctcaagctttgcggacaatagaa

SEQ ID NO: 28 - DNA, 37 bp, Artificial Sequence
  Primer
gccactagtggatccatgggaggaccagcttacgatt

SEQ ID NO: 29 - DNA, 37 bp, Artificial Sequence
  Primer
agcggtaccctcgaggtacttgagcctctcctttcta

SEQ ID NO: 30 - DNA, 37 bp, Artificial Sequence
  Primer
tggcgcgccactagtatgtcaggatgggacgaaggag

SEQ ID NO: 31 - DNA, 37 bp, Artificial Sequence
  Primer
accctcgaggtcgacgtaagctttgcggacaatagaa

SEQ ID NO: 32 - DNA, 20 bp, Artificial Sequence
  Primer
caccatgccgtgtggggagg

SEQ ID NO: 33 - DNA, 30 bp, Artificial Sequence
  Primer
tctagaaagttcatttccattcacacattt

SEQ ID NO: 34 - DNA, 23 bp, Artificial Sequence
  Primer
actctccacgaaataccactttg

SEQ ID NO: 35 - DNA, 24 bp, Artificial Sequence
  Primer
gtaggatgttgagggactgactcg

SEQ ID NO: 36 - DNA, 20 bp, Artificial Sequence
  Primer
cggctgtttaacttcgcttc

SEQ ID NO: 37 - DNA, 20 bp, Artificial Sequence
  Primer
cacacgccaagaaacagtga

SEQ ID NO: 38 - DNA, 32 bp, Artificial Sequence
  Primer
caaggtaccaagcttaatgccgtgtggggagg

SEQ ID NO: 39 - DNA, 32 bp, Artificial Sequence
  Primer
tgcggccgcatgcatttaaagttcatttccat

SEQ ID NO: 40 - DNA, 410 bp, Arabidopsis thaliana
  misc_feature: ETG1 promoter sequence
cttatgaaaaaactaaatatatgaaaacctaattcctctgttgtaaattcgtcatgtttagcttcatagatctctgtttctgtgtttgtgagtctggggtttgacttttaacaaatcttaagaccaagatggtcagaggatcatatatgccaaatgaattagtttattatacacatgaaacgacatcgtatggagaggggtccaaaacggtgcgttggaagttggagaatgggcatggataatggcgggaataacctaggagatatggcgggaaaggcccaactatacacacttgacattgcccttaattcgtgtttgagcaaagactcaatctttcatctctagattcttggcgattctcgctacatagtactttggtaaagagtgaaatcaaaggttggttttgagat

(19) United States
(12) Patent Application Publication
(10) Pub. No.: US 2011/0229491 A1
(43) Pub. Date: Sep. 22, 2011

(54) MINICHROMOSOME MAINTENANCE COMPLEX INTERACTING PROTEIN INVOLVED IN CANCER

(76) Inventors: Lieven De Veylder, Drongen (BE); Geert Berx, Linter-Wommersom (BE); Naoki Takahashi, Nara (JP); Mauricio Quimbaya, Gent (BE); Eric Raspe, Mouscron (BE)

(21) Appl. No.: 12/737,012

(22) PCT Filed: May 29, 2009
(86) PCT No.: PCT/EP2009/056658

§ 371 Date: May 19, 2011

(30) Foreign Application Priority Data
May 29, 2008 (US) 61130552

(51) Int. Cl. A61K 39/395; A61P 35/00; C40B 30/00; A61K 38/02; G01N 33/68; A61K 31/7088

(52) U.S. Cl. 424/172.1; 506/7; 514/19.3; 436/86; 514/44 A

(57) ABSTRACT

The present invention relates to a protein that is interacting with the minichromosome maintenance complex in eukaryotes. More specifically, the invention relates to the use of a protein, interacting with the minichromosome complex, and the gene encoding this protein in diagnosis, prognosis and treatment of cancer.

### CROSS-REFERENCE TO RELATED APPLICATIONS

This patent application is a national phase entry under 35 U.S.C. §371 of PCT International Patent Application PCT/EP2009/056658, filed May 29, 2009, designating the United States and published in English as International Patent Publication WO 2009/144311 A1 on Dec. 3, 2009, which claims the benefit, under Article 8 of the PCT, of U.S. Provisional Patent Application No. 61/130,552, filed May 29, 2008, the entire disclosure of each of which is hereby incorporated herein by this reference.

### FIELD OF THE INVENTION

The present invention relates to a protein that is interacting with the minichromosome maintenance complex in eukaryotes. More specifically, the invention relates to the use of a protein, interacting with the minichromosome complex, and the gene encoding this protein in diagnosis, prognosis and treatment of cancer.

### BACKGROUND

All eukaryotic cells replicate their nuclear DNA in a conserved manner, whereby the parent molecule is unwound and each DNA strand becomes the template for nascent DNA synthesis. Coordination of DNA replication with growth and development is essential for survival, as mistakes made during DNA replication can result in apoptosis, growth defects, or oncogenesis. Because of its essential role during development, it is not surprising to see that the molecular machinery controlling DNA replication is highly conserved among organisms. Despite the many years of evolution that separate animals from plants, both organisms use the same E2F/DP pathway to regulate entry into S phase by controlling transcriptional induction of genes required for cell cycle progression and DNA replication. The genome of Arabidopsis contains six E2Fs (E2Fa, E2Fb, E2Fc, E2Fd/DEL2, E2Fe/DEL1, and E2Ff/DEL3) and two DPs (DPa and DPb) (Inzé and De Veylder, 2006). Three E2F proteins (E2Fa-c) bind DNA through the consensus E2F binding site by forming heterodimers with DP proteins. Both E2Fa and E2Fb operate as transcriptional activators, whereas E2Fc functions as a repressor (De Veylder et al., 2002; del Pozo et al., 2002). The remaining Arabidopsis E2Fs (E2Fd/DEL2, E2Fe/DEL1 and E2Ff/DEL3) contain duplicated DNA-binding domains, allowing binding to consensus E2F sites as a monomer (Kosugi and Ohashi, 2002; Ramirez-Parra et al., 2004; Vlieghe et al., 2005).

Both in mammals and Arabidopsis, numerous E2F target genes have been identified using microarrays, chromatin immunoprecipitations, and in silico analyses (Ramirez-Parra et al., 2003; Dimova and Dyson, 2005; Vandepoele et al., 2005). These genes encode proteins active during DNA replication, mitosis, DNA checkpoint control, apoptosis, or differentiation. Remarkably, almost every gene encoding for a protein involved in licensing for DNA replication is transcriptionally controlled by E2F transcription factors, such as the origin recognition complex (ORCs), CDC6, minichromosome maintenance complex (MCMs), and CDT1 genes (Vandepoele et al., 2005). Licensing for DNA replication in eukaryotes is initiated by the formation of the pre-replicative complex (pre-RC) at replication origins (Gillespie et al., 2001; Bell and Dutta, 2002; Diffley and Labib, 2002). First, ORC proteins bind to DNA during the early G1-to-S phase of the cell division cycle. Then, CDC6 binds to these ORC-DNA sites, an event that is followed quickly by binding of CDT1. Finally, replication origins are licensed by loading the MCM complex to form a pre-RC. The MCM complex is a heterohexamer composed of MCM2 to 7 and is likely a component of the helicase that unwinds DNA during replication (Tye and Sawyer, 2000; Labib and Diffley, 2001; Forsburg, 2004).

Once the formation of the pre-RC has been completed by the loading of MCMs, the DNA is primed for replication through the action of two conserved protein kinases, cyclin-dependent protein kinase (CDK) and Cdc7-Dbf4 (Dbf4-dependent kinase, DDK), resulting into the recruitment of additional replication factors to form the pre-initiation complex (pre-IC) (Kamimura et al., 2001; Masumoto et al., 2002; Takayama et al., 2003; Kanemaki et al., 2006). Loading of the pre-IC onto the origins activates the MCM helicases and recruitment of DNA polymerases, resulting into the initiation of DNA synthesis (Zou and Stillman, 2000). After origin firing, some of the initiation factors move with replication forks to support the elongation step of DNA synthesis (Aparicio et al., 1997; Takayama et al., 2003; Gambus et al., 2006; Kanemaki et al., 2006). Members of the MCM complex, particularly MCM2, have been described as markers for screening, surveillance and prognosis of cancer (Giaginis et al., 2008).

By studying E2F target genes in Arabidopsis, we have identified ETG1 (At2g40550) as a novel E2F target gene, being directly controlled by the E2Fa and E2Fb transcription factors. ETG1 null mutants display a slower cell cycle progression. Genetic analysis and transcriptional up-regulation of the PARP2, WEE1 and RAD51 genes indicated that this cell cycle delay originates from the activation of the DNA replication checkpoint. ETG1 is demonstrated to associate with the DNA replication complex, suggesting that the activation of the DNA replication checkpoint in ETG1-deficient plants originates from impaired DNA replication. Surprisingly, the absence of a functional ETG1 allele in a wee1 or atr mutant background has a profound impact on plant development, illustrating that the DNA replication checkpoint aids to the survival of ETG1-deficient plants.

Even more surprisingly, knock down mutants of the human ETG1 homologue (C10orf119) resulted in a similar phenotype, indicating that the gene is involved in the development of mammalian tumors. This is specifically surprising, as Sakwe et al. (2007) indicated that C10orf119 (MCM-BP) binds to the MCM complex, but only in absence of the most relevant tumor marker MCM2.

### DISCLOSURE OF THE INVENTION

A first aspect of the invention is the use of ETG1 or an ETG1 ortholog for the diagnosis and/or prognosis of cancer. An ortholog, as used here, means a sequence with a similar, preferably an identical, function as the reference protein, and a detectable homology (expressed as percentage identity) with the reference sequence. ETG1 orthologs include, but are not limited to, rice (Oryza sativa; Os01g0166800), human (C10orf119), mouse (1110007A13Rik), Xenopus (CAJ81286), Drosophila (CG3430) and fission yeast (SPAC1687.04) orthologs. Preferably, the ETG1 ortholog is the human ortholog C10orf119 (accession NP_{—}079110). The human ETG1 gene is located on chromosomal position 10q26.11. This particular region shows high frequency of loss of heterozygosity in human meningiomas and colorectal cancers (Mihaila et al., 2003; Karoui et al., 2004). This type of loss of heterozygosity is generally regarded as a hallmark for the localization of a tumor suppressor.

The “use” as meant herein is any use of the nucleic acid or protein and may be, as a non-limiting example, the genomic DNA for the detection of mutation, the mRNA or derived cDNA for the analysis of the expression, or the protein for the analysis of translated protein. Methods for mutation and snp analysis, expression analysis and detection and quantification of protein are known to the person skilled in the art. Abnormal chromosome content is the most common characteristic of human solid tumors. Preferably, the cancer is a cancer originating from a chromatin cohesion defect. Even more preferably, the cancer is selected from the group consisting of Seminoma, Colon carcinoma, Cervical cancer, Acute Myeloid carcinoma, Wilson tumor, Oligodendroglioma, Renal Carcinoma, Prostate carcinoma and Breast carcinoma.

Another aspect of the invention is the use of an ETG1 ortholog to treat cancer. Preferably, the use is the modulation of the expression. “Modulation,” as used herein, may be under- or over-expression. A knock down may be realized by, as a non-limiting example, the expression of RNAi. Alternatively, the level of protein may be modulated by a specific interaction, such as the binding of an antibody. Preferably, the ETG1 ortholog is the human ortholog C10orf119. One preferred embodiment is a modulation whereby modulation is a down-regulation in the cancers selected from the group consisting of Seminoma, Colon carcinoma, Acute Myeloid carcinoma, Wilson tumor, and Oligodendroglioma. Another preferred embodiment is a modulation whereby modulation is an up-regulation in the cancers selected from the group consisting of Renal Carcinoma, Prostate carcinoma and Breast carcinoma.

Still another aspect of the invention is the use of ETG1 or an ETG1 ortholog to screen compounds interfering with the interaction of ETG1 or an ETG1 ortholog with the MCM complex. Preferably, the compound is a compound that interferes with the interaction of ETG1 or an ETG1 ortholog with MCM2, MCM3, MCM4, MCM5, MCM6 and/or MCM7. “Interfering,” as used herein, can be either positive or negative, making the interaction stronger or disturbing the interaction. Methods to study protein-protein interactions (and the effect on compounds on those interactions) are known to the person skilled in the art and include, but are not limited to, two yeast hybrid studies, mappit (Tavernier et al., 2002) and reverse mappit (Lemmens et al., 2006).

Still another aspect of the invention is the use of a compound interfering with the interaction of ETG1 or an ETG1 ortholog with the MCM complex, isolated according to the invention, to treat cancer.

### BRIEF DESCRIPTION OF THE FIGURES

FIG. 1: Molecular and phenotypic analysis of ETG1-deficient plants. (A) The exon (boxes) and intron (lines) structure of ETG1. Coding and non-coding regions are shown as black and white boxes, respectively. White triangles indicate T-DNA insertion sites. Arrows indicate primer positions used for real-time RT-PCR analysis. (B) Real time RT-PCR analysis of ETG1 expression in wild-type (WT), etg1-1 and etg1-2 plants. RT-PCR was performed using total RNA prepared from first leaves of nine-day-old plants. All values were normalized against the expression level of the ACTIN2 gene. (C) Seedling phenotypes of 21-day-old wild-type (WT) and etg1-1 plants. (D) Ploidy level distribution of the first leaves of three-week-old wild-type (col-0), etg1-1 and etg1-2 plants as measured by flow cytometry. Data represent average ±SD (n=5). (E) Drawing-tube image of the first leaves of three-week-old wild-type (WT; left) and etg1-1 (right) plants. Bar indicates 100 μm. (F-H) Leaf growth of the first leaf pair of wild-type (WT), etg1-1 and etg1-2 plants. Leaf blade area (F), Epidermal cell number on the abaxial side of the leaf (G), and epidermal cell size on the abaxial side of the leaf (H). Data represent average ±SD (n=5). (I-J) Adult phenotype of wild-type (WT) and etg1-1 plants. The plants were photographed five weeks after germination. (J) shows magnification of leaves in wild-type and etg1-1 plants.

FIG. 2: Kinematic analysis of first leaf pair of wild-type (WT) and etg1-1 plants. (A) Leaf blade area; (B) average cell area on abaxial side of the leaf; (C) number of cells on abaxial side of leaf; and (D) cell division rate. Leaves were harvested at the indicated time points. Data represent average ±SD (n=5).

FIG. 3: ETG1-depletion leads to a G2 arrest. (A) Ploidy level distribution of the first leaves of eight-day-old wild-type (WT) and etg1-1 plants as measured by flow cytometry. (B) Ratio of 4C/2C cells by flowcytometry using first leaves of eight-day-old wild-type (WT) and etg1-1 plants. Data represent average ±SD (n=3). (C) Real time RT-PCR analysis of CDKB1;1 and CYCB1;1 expression in wild-type (WT) and etg1-1 plants. RT-PCR was performed using total RNA prepared from nine-day-old seedlings. All values were normalized against the expression level of the ACTIN2 gene.

FIG. 4: ETG1 gene expression is regulated by the E2Fa transcription factor. (A) Sequence of the ETG1 promoter showing the presence of two E2F binding sites at −158 by (I) and -136 (II) from the ATG translation start codon (SEQ ID NO:40). (B) ChIP assays in eight-day-old Arabidopsis plants using antibodies specific for E2Fa, E2Fb, E2Fc and DELL Semiquantitative PCR was used to estimate the relative enrichment of genomic fragments of ETG1 promoter. (C) Real time RT-PCR analysis of ETG1 expression in wild-type (WT) and E2Fa/DPa overexpressing plants. RT-PCR was performed using total RNA prepared from six-day-old plants. All values were normalized against the expression level of the ACTIN2 gene. (D) Histochemical localization of GUS activity in transgenic six-day-old seedlings carrying the wild-type (col-0), either one (ΔI or ΔII) or both (ΔI,II) of the E2F elements ETG1 promoters fused to GUS gene. The shoot (upper) and the root apical regions (bottom).

FIG. 5: ETG1 assembles into the replisome and is essential for DNA replication. (A) Subcellular localization of ETG1. The full length ETG1-eGFP fusion protein is localized in nucleus. (B) ETG1 interacts with MCM5 in yeast. Yeast PJ69-4a cells were transformed with a plasmid encoding a GAL4 DNA binding domain-ETG1 and -MCM5 fusion (GAL4-DBD-ETG1 and -MCM5), respectively. Yeast PJ69-4-alfa cells were transformed with GAL4 activation domain-ETG1, -MCM5 and -GUS fusion as negative control (GAL4-AD-ETG1, -MCM5 and -GUS). Interactions between fusion proteins were assayed by mating method. Diploid strains were spotted on plates with (+His, positive control) or without (-His) histidine. (C) BiFC assay of ETG1 interaction with MCM5 in plant. Tobacco epidermal cells were transfected with combination of constructs encoding indicated fusion proteins. YFPN, the fragment containing amino acid residues 1-154 of YFP; YFPC, the fragment containing amino acid residues 155-238 of YFP. Arrowheads show nuclei. (D) Subcellular localization of MCM5. The full length GFP-MCM5 fusion protein is localized in nucleus and cytoplasm. (E) BrdU incorporation of the wild-type (col-0) and etg1-1. Three-day-old seedlings were soaked in BrdU solution for the indicated periods, and genomic DNA was extracted from them. The amounts of BrdU incorporation were determined by ELISA using an anti-BrdU antibody. Data represent average ±SD (n=3).

FIG. 6: Loss of ETG1 induces expression of DNA replication checkpoint and DNA repair genes. (A) Real time RT-PCR analysis of WEE1 and RAD51 expression in wild-type (white bars) and etg1-1 (black bars) plants. RT-PCR was performed using total RNA prepared from nine-day-old seedlings. All values were normalized against the expression level of the ACTIN2 gene. (*) and (**) indicate significant statistical differences by t-test (p<0.05 and p<0.01, respectively) between wild-type and etg1-1. Data represent average ±SD (n=3). (B-F) Histochemical localization of GUS activity in transgenic six-day-old PARP2::GUS (B), PARP2::GUS crossed with etg1-1 (C), PARP2::GUS grown on MS agar plate with 1 μg/ml bleomycin (D), WEE1::GUS (E) and WEE1::GUS crossed with etg1-1 plants. All seedlings were grown on MS agar plate except for (C).

FIG. 7: ETG1 depletion activates ATR/WEE1 DNA replication stress checkpoints. (A-H) Seedlings phenotype of 21-day-old wild-type (col-0) (A), etg1-1 (B), wee1-1 (C), atr-2 (D), etg1-1/wee1-1 (E, F) and etg1-1/atr-2 (G, H) grown on MS plate. (F) and (H) show magnification of etg1-1/wee1-1 and etg1-1/atr-2 plants. Bars: 5 mm (A-E, G) and 1 mm (F and H). (I-Q) Scanning electron micrographs of the 14-day-old whole seedlings (I-K), leaf epidermal cells (L-N) and trichome (O-Q). (I, L, O) wild-type; (J, M, P) etg1-1/wee1-1, and (K, N, Q) etg1-1/atr-2. Bars: 500 μm (A-C), 50 μm (D-F, H, and I) and 100 μm (G).

FIG. 8: Up-regulation of mitosis-specific expression genes in etg1. (A) G0 analysis of 121 up-regulated genes in etg1 in the ATH1 microarray experiment. (B) Enrichment of M-phase-specific genes in the etg1 transcriptome dataset. (C) Cell cycle phase comparison of up-regulated genes in etg1, UV-B-treated and bleomycin-treated plants. S-phase (red), G2-phase (blue), M-phase (yellow), and G1 phase (green) specific expression genes.

FIG. 9: ETG1 is required for establishment of sister chromatid cohesion. (A) Scheme of chromosome 1 with a sequence cloned in BAC T2P11/T7N9, BACF11P17 and a 178-bp centromere-specific sequence (pAL). (B-F) One FISH signal (B, pairing of both homologs) or two FISH signals (C, D) per BAC (T2P11/T7N9) were regarded as positional alignment at corresponding region, indicating sister chromatids are aligned. Three (E) or four (F) signals were considered to indicate sister-chromatid separation. (G) Percentage of sister-chromatid alignment/separation frequencies analyzed in wild-type (col-0) and etg1 after FISH with labeled BAC from chromosome 1. BAC T2P11/T7N9 were tested in 4C leaf nuclei.

FIG. 10: Morphology of sub-confluent cultures of wild-type MCF7 cells.

FIG. 11: Aberrant phenotypes (multi-nucleated and giant cells) observed in hETG1 knocked-down (pictures above) and over-expressing (pictures below) MCF7 cell cultures.

FIG. 12: Examples of giant and multi-nucleated cells observed upon over-expression of hETG1. The hETG1 protein is in the green channel and β-catenin protein is in the red channel.

FIG. 13: Flow cytometry analysis of wild-type and hETG1 knock-down (158067-Const) cell lines. Knock-down cell line shows a depletion of 11% of G1 cells, correlated with a 10% increment in G2 cells. A minor group of cells (1%) displayed a DNA content higher than 4C.

FIG. 14: Up-regulation of G2-M markers upon hETG1 knock-down. Cells were harvested after three days of growing, total RNA was extracted and cDNA was synthesized. Relative expression values were normalized against TBP and UBC. The expression levels in control MCF7 cells were arbitrarily set to 1.

FIG. 15: Up-regulation of MAD3 kinetochore marker upon hETG1 knock-down. Cells were harvested after three days of growing, total RNA was extracted and cDNA was synthesized. Relative expression values were normalized against TBP and UBC. The expression levels in control MCF7 cells were arbitrarily set to 1.

FIG. 16: Karyotypes for sister chromatids problems, detecting chromosomes with totally detached sister chromatids (enclosed in green circles).

FIG. 17: Expression analysis of a series of 56 primary human breast cancers. Relative ETG1 expression levels (average of ten samples with low expression set to 1) were depicted ranking low to high.

FIG. 18: Distribution of primary breast cancers according to ER status and ETG1 expression. Tumor samples were grouped in quartiles based on ETG1 expression levels from low Q1 to high Q4.

### DETAILED DESCRIPTION OF THE INVENTION

### Examples

### Example 1

### The Loss of ETG1 Suppresses Cell Division

Previously, we identified 70 conserved plant E2F target genes (Vandepoele et al., 2005). This list holds 40 known regulators of DNA replication and chromatin dynamics, as well as 21 genes with unknown function. To identify among the latter novel S phase regulatory genes, we performed a phenotypic screening using T-DNA insertion lines obtained from the Salk Institute Genomic Analysis Laboratory (http://signal.salk.edu/cgi-bin/tdnaexpress). One of these T-DNA insertion mutants showed an endoreduplication phenotype. We designated this T-DNA insertion mutant as E2F target gene 1 (etg1; At2g40550).

To address the role of ETG1 in plant growth and development, we analyzed the effect of loss of function of ETG1 in detail using two independent T-DNA insertion lines. Plants were grown under long-day conditions (16 hours/light, 8 hours/darkness) at 22° C. on 0.5×MS agar plate (Valvekens et al., 1988). The etg1-1 (SALK_{—}071046) and etg1-2 (SALK_{—}145460) alleles were found in the Salk Institute Genomic Analysis Laboratory engine (http://signal.salk.edu/cgi-bin/tdnaexpress). These seeds were acquired from the ABRC.

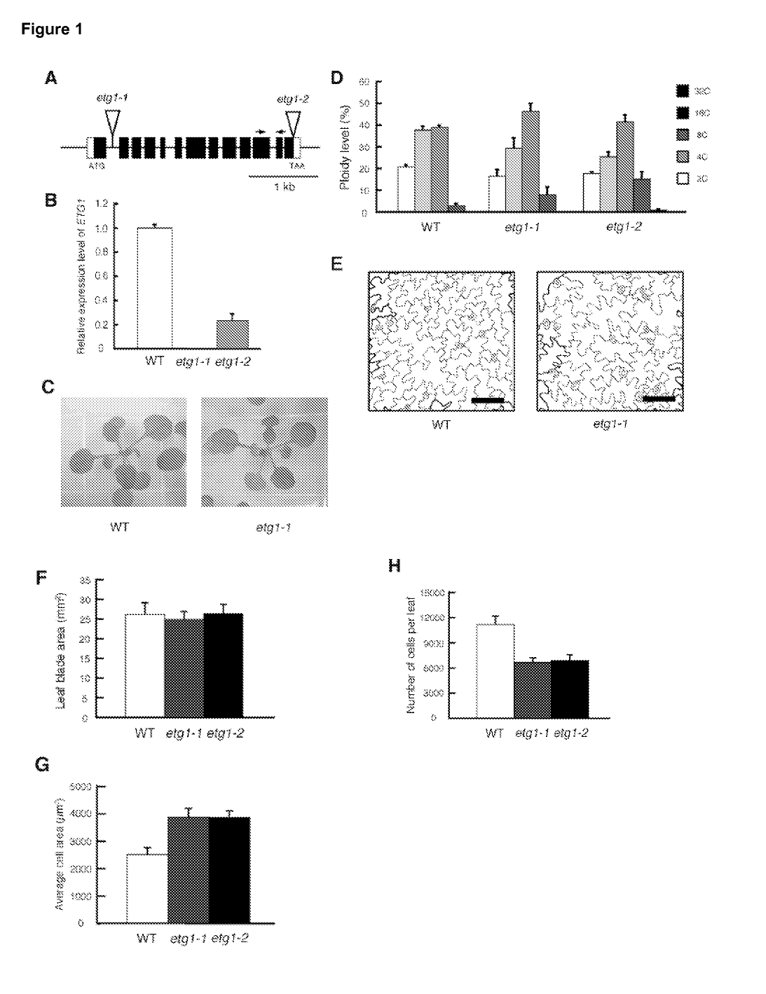

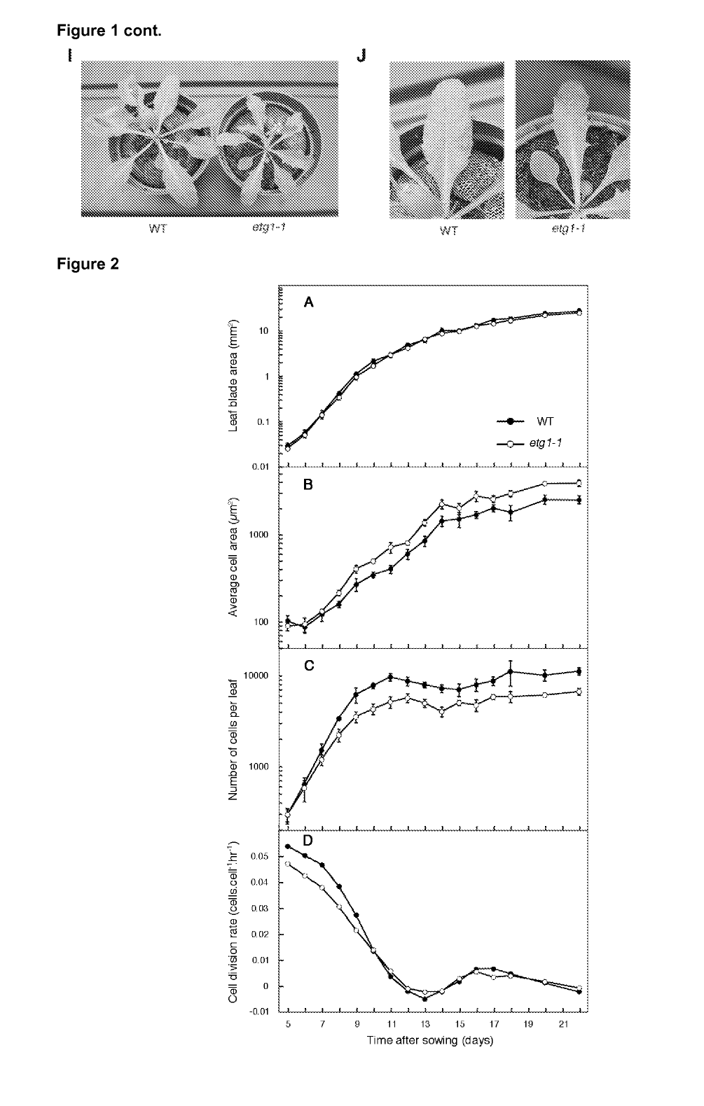

To screen for homozygous insertion alleles, the following primer pairs were designed: 5′-AGACCAAGATGGTCAGAGGATC-3′ (SEQ ID NO:1) and 5′-ACTGGAACACAGTAAAGCAAGC-3′ (SEQ ID NO:2) for etg1-1, and 5′-AAATTAACCGGAATGGGTTTG-3′ (SEQ ID NO:3) and 5′-ATGACTCAGATTGATGCCTGG-3′ (SEQ ID NO:4) for etg1-2. The T-DNA was inserted in the first intron (etg1-1; SALK_{—}071046) or last exon (etg1-2; SALK_{—}145460) of the ETG1 gene (FIG. 1, Panel A), respectively. ETG1 transcripts were not detected in the etg1-1 mutant, whereas 80% reduction in transcript level was observed in etg1-2, compared to control plants (FIG. 1, Panel B). In etg1 mutant seedlings, plant growth appeared macroscopically normal (FIG. 1, Panel C). However, by comparing the ploidy level of wild-type plants with etg1-1 and etg1-2 mutants, a significant change in the distribution of the C values was found. etg1 mutants leaves contained an increased population of cells with an 8C and 16C DNA ploidy level, demonstrating that deficiency for ETG1 stimulated endoreduplication (FIG. 1, Panel D). When comparing the first pair of leaves from wild-type and etg1 mutant plants at maturity, the leaf blade area was found to be almost identical for both genotypes (FIG. 1, Panel F). By contrast, a significant increase in the average abaxial pavement cell area was observed in the mutant plants (FIG. 1, Panels E and G), accompanied with a decrease in the number of cells per leaf (FIG. 1, Panel H).

At the stage of bolting, younger leaves showed a slightly elongated and serrated leaf phenotype (FIG. 1, Panels I and J), resembling the phenotype observed for plants in which cell division is inhibited by ectopic expression of the CDK inhibitory KRP2 gene (De Veylder et al., 2001). In addition, the root growth rate of the mutant plants was significantly reduced, suggesting an inhibition of the cell cycle in etg1 mutant plants.

To study the effect of loss of ETG1 function on cell cycle progression in more detail, we performed a leaf kinematic growth analysis. Kinematic analyses of leaf growth was performed as described by Boudolf et al. (2004) and De Veylder et al. (2001). Leaf growth of etg1 and Col-0 was analyzed on five plants from 5 to 22 DAS by measuring the total leaf blade area of all cells from the abaxial epidermis drawn with a drawing tube attached to the microscope, the total number of cells. The average cell area was determined from the number and total area of drawn cells, and the total number of cells per leaf was calculated by dividing the leaf area by the average cell area (averaged between the apical and basal positions).

Finally, the average cell division rate for the whole leaf was determined as the slope of the log 2-transformed number of cells per leaf, which was done using five-point differentiation formulas (Erickson, 1976). Subsequently, seedlings were fixed in 100% ethanol overnight, replaced by lactic acid for microscopy. When wild-type and etg1-1 were compared, significant differences were observed (FIG. 2). Leaf blade area was similar in the wild-type and etg1-1 plants during the whole period of leaf development (FIG. 2, Panel A). However, the average cell area, which initially was about 100 μm² in both plants, increased significantly faster in the etg1-1 mutant. The average surface area of etg1-1 cells was 155% of those of wild-type cells at maturity (3,880±320 versus 2,500±255 μm², respectively; FIG. 2, Panel B). Simultaneously, the number of epidermal cells of etg1-1 was only about 60% of these of wild-type (6,650±530 versus 11,170±1,017 cells, respectively; FIG. 2, Panel C).

Until nine days after sowing, the average cell division rate for the whole leaf, calculated on the basis of the increase of cell numbers over time, were constantly lower in the etg1-1 mutant than wild-type leaves (FIG. 2, Panel D). The average cell cycle duration time between days 5 and 9, estimated as the inverse of the cell division rate, was significantly longer in the etg1-1 mutant compared to wild-type plants (25.3 hours versus 21.1 hours, respectively). In summary, these data illustrate that ETG1-deficient plants suffer from a cell cycle delay, resulting in a reduction in total cell number. This reduction in cell number is offset by an increase in cell size, resulting into a nearly identical leaf size.

### Example 2

### Loss of ETG1 Function Causes a G2 Cell Cycle Arrest

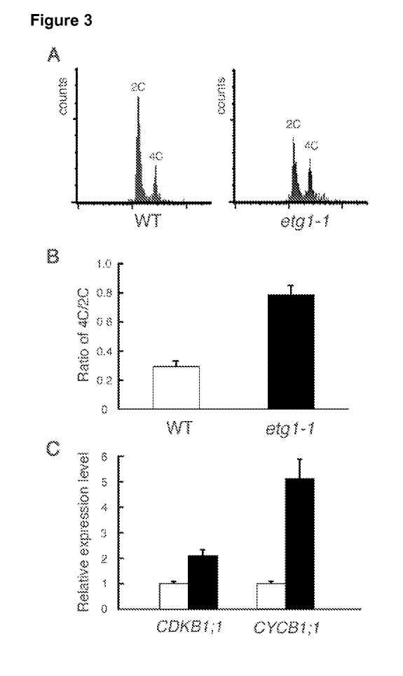

To pinpoint the cell cycle arrest point, we measured the ratio of 4C/2C cells by flow cytometry using eight-day-old leaves. As at this time point, leaf cells of both genotypes are dividing (FIG. 2, Panels C and D); consequently, 2C and 4C cells represent G1 and G2 cells, respectively. The flow cytometry was performed on plants grown in Petri dishes filled with 0.5×MS agar as described by Boudolf et al. (2004). Three biological and two technical replicates were used. By comparing the ploidy level of wild-type and etg1-1, a significant increase in the ratio of 4C/2C cells was observed in etg1-1 plants (0.79±0.04 versus 0.29±0.06 in etg1-1 and wild-type plants, respectively; FIG. 3, Panels A and B). These data indicate an inhibition of the G2-to-M transition in the etg1-1 mutant.

As a confirmation, the expression levels of a number of cell cycle marker genes were analyzed by real-time RT-PCR. RNA was extracted from Arabidopsis tissues with RNEASY® Plant Mini Kit (Qiagen). First-strand cDNA was prepared from total RNA with the Superscript™ III First-Strand Synthesis System (Invitrogen) and according to the manufacturer's instructions. Quantitative PCR was performed with the LIGHTCYCLER® 480 SYBR Green I Master (Roche) with 100 nM primers and 0.1 μg of RT reaction product. Reactions were run and analyzed on the LIGHTCYCLER® 480 Real-Time PCR System (Roche) according to the manufacturer's instructions. All quantifications were normalized to ACTIN2 cDNA fragments amplified under the same conditions. Quantitative reactions were done in triplicate and averaged. Primers used were 5′-GGCTCCTCTTAACCCAAAGGC-3′ (SEQ ID NO:5) and 5′-CACACCATCACCAGAATCCAGC-3′ (SEQ ID NO: 6) for ACTIN2, 5′-TTGCAACCAGGCACCTTGAA-3′ (SEQ ID NO:7) and 5′-CAAATCGGCGGGCATTATGT-3′ (SEQ ID NO:8) for ETG1, 5′-TGGTGCTGGACATTTCAGTCGG-3′ (SEQ ID NO:9) and 5′-CAAGAGCTTGCACTTCCATCATAG-3′ (SEQ ID NO:10) for WEE1, 5′-CGAGGAAGGATCTCTTGCAG-3′ (SEQ ID NO:11) and 5′-GCACTAGTGAACCCCAGAGG-3′ (SEQ ID NO:12) for RAD51, 5′-CTCAAAATCCCACGCTTCTTGTGG-3′ (SEQ ID NO:13) and 5′-CACGTCTACTACCTTTGGTTTCCC-3′ (SEQ ID NO:14) for CYCB1;1, and 5′-CTCGAGATGGACGAAGAAGG-3′ (SEQ ID NO:15) and 5′-CGACGCAGAGTAATCGAACA-3′ (SEQ ID NO:16) for CDKB1;1. A substantial increase in the transcript level of the G2-to-M-phase-specific cyclin CYCB1;1 and CDKB1;1 genes were seen in the etg1-1 mutant (FIG. 3, Panel C). These data confirmed that the observed increase in the number of 4C cells is specifically due to a G2 arrest rather than a consequence of cells prematurely entering the endoreduplication cycle.

### Example 3

### ETG1 Transcript is Regulated by E2Fa and E2Fb Transcription Factors

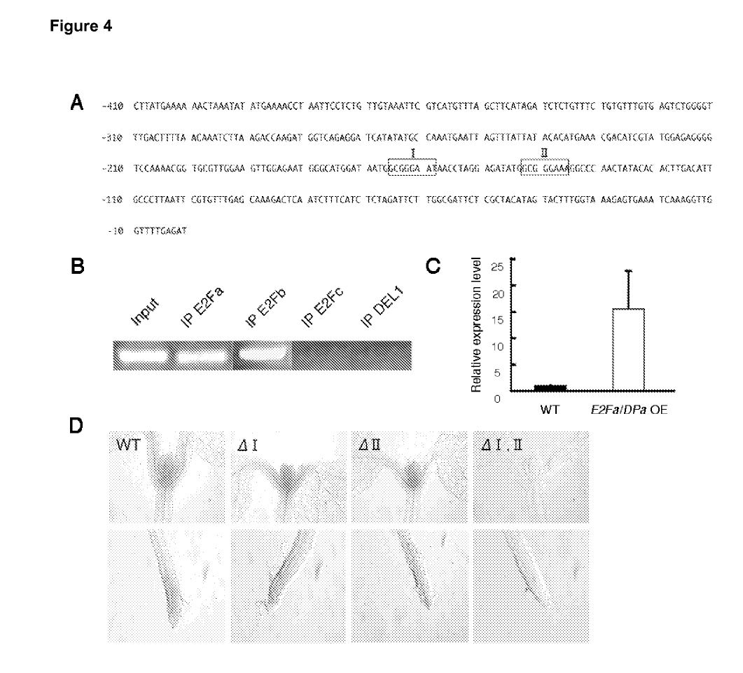

The ETG1 gene was originally identified by microarray analysis, comparing the transcriptome of wild-type Arabidopsis plants with that of plants ectopically expressing the heterodimeric E2Fa-DPa transcription, showing a strong up-regulation of ETG1 into the latter (Vandepoele et al., 2005). This induction was confirmed by quantitative real-time PCR analysis (FIG. 4, Panel C). To analyze whether the ETG1 gene is directly regulated by E2F transcription factors, we searched for the presence of consensus E2F binding sites in its putative promoter region. Two consensus E2F binding elements were found, ATTCCCGC (158 by upstream from the putative start codon) and TTTCCCGC (136 by upstream), both in a reverse orientation (FIG. 4, Panel A).

To address whether ETG1 is an E2F target gene in vivo, we performed chromatin immunoprecipitation (ChIP) experiments. Immunoprecipitations were performed using anti-E2Fa, -E2Fb, -E2Fc, and DEL1 antibodies. ChIP was performed according to Bowler et al. (2004) with few modifications. Briefly, 1 g of eight-day-old seedlings was harvested, rinsed in ddH₂O, and cross-linked in 1% formaldehyde for ten minutes. Cross-linking was stopped by addition of glycine to a final concentration of 0.125 M. Tissue was grinded and chromatin extracted. The chromatin solution was sonicated using a Branson 1200 sonifier. After preclearing, 10 μl of the appropriate antibodies was added to the chromatin solution and incubated overnight at 4° C. After collection of the immunoprecipitate with protein A agarose beads, beads were washed and immunocomplexes eluted. Cross-linking was reversed by incubation at 65° C. overnight. Proteinase K digestion was followed by phenol/chloroform extraction and ethanol precipitation. Recovered DNA was used in 25 cycle PCR reaction.

As shown in FIG. 4, Panel B, ETG1 promoter sequences were not recovered from the immunoprecipitates with either anti-DEL1 or anti-E2Fc antibodies. However, promoter fragments of the ETG1 gene were specifically amplified from the anti-E2Fa and -E2Fb immunoprecipitates. These results indicate that E2Fa and E2Fb can bind directly to the ETG1 promoter in vivo, likely participating in the regulation of its expression.

The regulation of the ETG1 promoter activity through its E2F consensus sites was further analyzed using transgenic plants expressing the β-glucuronidase (GUS) reporter gene under control of the ETG1 promoter. To define the contribution of each of the E2F binding sites, we deleted either one (ΔI or ΔII) or both (ΔI,II) of the E2F elements. The ETG1 promoter sequence was amplified from Arabidopsis genomic DNA by PCR with the FP-ETG1 (5′-GGGGACAAGTTTGTACAAAAAAGCAGGCTTCATATGAAAACCTAATTCCTCTG-3′ (SEQ ID NO:17)) and RP-ETG1 (5′-GGGGACCACTTTGTACAAGAAAGCTGGGTCG GTCAGACAATCGTAAGCTGGT-3′ (SEQ ID NO:18)) primers. Each E2F element in the ETG1 promoter were mutated by two-step overlap extension PCR with FP-ETG1, RPETG1 primers and promoter-specific primers, FPΔI-ETG1 (5′-ATGGATAATGAACCTAGGAGATATG-3′ (SEQ ID NO:19)) and RPΔI-ETG1 (5′-CTCCTAGGTTCATTATCCATGCCCATTC-3′ (SEQ ID NO:20)) for mutation of the first E2F element (I), and FPΔII-ETG1 (5′-AGGAGATATGGGCCCAACTATACACACTTG-3′ (SEQ ID NO:21)) and RPΔII-ETG1 (5′-TAGTTGGGCCCATATCTCCTAGGTT-3′ (SEQ ID NO:22)) for mutation of the second E2F element (II), respectively (FIG. 4, Panel A). Both E2F elements in the ETG1 promoter were mutated by PCR using PCR fragments of ΔI-ETG1 promoter with FP-ETG1, RP-ETG1, FPΔII-ETG1 and RPΔII-ETG1 primers.

Each PCR fragment was cloned into pDONR201 entry vector by BP recombination reaction and subsequently transferred into the pKGWFS7 destination vector (Karimi et al., 2002) by LR recombination reaction, resulting in a transcriptional fusion between the ETG1 promoter and the eGFP-GUS fusion gene. All constructs were transferred into the Agrobacterium tumefaciens C58C1RifR strain harboring the plasmid pMP90. The obtained Agrobacterium strains were used to generate stably transformed Arabidopsis with the floral dip transformation method (Clough and Bent, 1998). Seedlings were stained on multiwell plates (Falcon 3043; Becton Dickinson). GUS assays were performed as described by Beeckman and Engler (1994). Samples mounted in lactic acid were observed and photographed with a differential interference contrast microscope (Leica).

More than five independent transgenic lines were analyzed per gene construct, all showing identical results. In six-day-old seedlings, high levels of ETG1 expression were observed in the shoot apical and root meristem (FIG. 4, Panel D). This expression pattern matches that one of the E2Fa and DPa genes (De Veylder et al., 2002). Deletion of either one of the E2F binding elements (ΔI or ΔII) led to GUS activity pattern identical to that of plants carrying the wild-type ETG1 promoter (FIG. 4, Panel D). In contrast, deletion of both E2F binding elements resulted into a drastic decrease in promoter activity (FIG. 4, Panel D). These results suggest that E2Fa/E2Fb bind both E2F consensus elements in the ETG1 promoter and regulates its expression in dividing tissues.

### Example 4

### ETG1 is Nuclear Protein Conserved in Eukaryotes

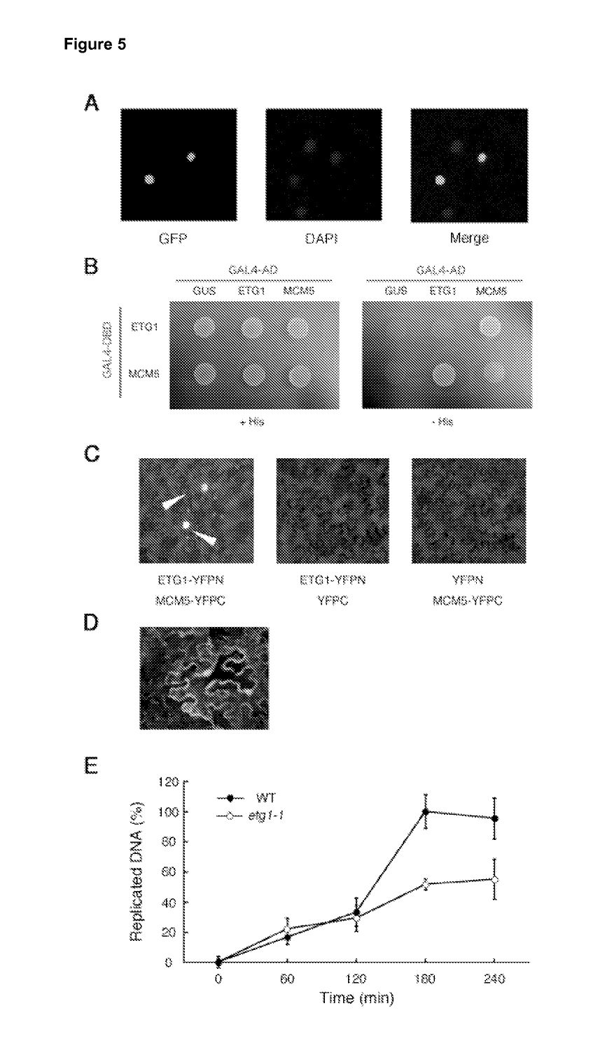

The ETG1 gene encodes for a protein of 589 amino acid residues (At2g40550; genbank accession AAY25444). ETG1 is a singleton in Arabidopsis. When searching for similar proteins by sequence comparison, no identification was found with any other functional annotated protein, neither a specific amino acid domain could be identified with the exception for a putative nuclear localization signal, PFKKMKV (SEQ ID NO:23) (amino-acids 184-190), suggesting that ETG1 resides in the nucleus. To investigate the subcellular localization of ETG1, an ETG1:eGFP (GFP for enhanced green fluorescent protein) fusion protein was transiently expressed in tobacco leaf epidermal cells. ETG1:eGFP fluorescence was observed in the nucleus only, illustrating that ETG1 is a nuclear protein (FIG. 5, Panel A).

ETG1 orthologous proteins were found in rice (Oryza sativa; Os01g0166800), human (C10orf119), mouse (1110007A13Rik), Xenopus (CAJ81286), Drosophila (CG3430) and fission yeast (SPAC1687.04) (Supplemental FIG. 2). In every case, a putative E2F consensus element could be identified in the corresponding putative promoter region, indicating that ETG1 is highly conserved E2F target gene. Interestingly, fission but no budding yeast ortholog could be identified.

### Example 5

### ETG1 is a Component of the Replisome Complex and Essential for DNA Replication

Patterns of coexpression can reveal networks of functionally related genes and provide deeper understanding of processes requiring multiple gene products (Stuart et al., 2003; Wei et al., 2006). To predict ETG1 function, we searched for genes coexpressed with ETG1 using the ATTED-II coexpression database (Obayashi et al., 2007). This search revealed that ETG1 is highly coexpressed with genes encoding DNA replication proteins, such as minichromosome maintenance family proteins (MCM2, 3, 4, 5 and 7), proliferating cell nuclear antigen proteins (PCNA1 and 2), DNA primase small subunit protein, and DNA polymerase alpha subunits (Table 1). Moreover, when searching for proteins interacting with orthologous ETG1 proteins, using the BioGRID protein interactions database (http://www.thebiogrid.org/index.php), we found that the Drosophila ortholog CG3430 interacted with MCM5 protein. An identical interaction between the Arabidopsis ETG1 and MCM5 (At2g07690) proteins was demonstrated using the yeast two-hybrid system (FIG. 5, Panel B). Yeast two-hybrid bait and prey vectors were obtained through recombinational GATEWAY cloning (Invitrogen). The ETG1 and MCM5 open reading frames were recombined into the pDEST22 and pDEST32 vectors (Invitrogen) by an LR reaction, resulting in translational fusions between the open reading frames and the GAL4 transcriptional activation and GAL4 DNA binding domains, respectively. Plasmids encoding the baits and preys were transformed into the yeast strain PJ69-4-alfa (MATalpha; trp1-901, leu2-3,112, ura3-52, his3-200, gal4Δ, gal80Δ, LYS2::GAL1-HIS3, GAL2-ADE2, met2::GAL7-lacZ) and PJ69-4a (MATa; trp1-901, leu2-3,112, ura3-52, his3-200, gal4Δ, gal80Δ, LYS2TGAL1-HIS3, GAL2-ADE2, met2TGAL7-lacZ) by the LiAc method (Gietz et al., 1992), and plated on SD plates without Leu and on SD plates without Trp for two days at 30° C., respectively. Interactions between fusion proteins were assayed by mating method. Diploid strains were transferred to SD plates without Leu and Trp (as a control) and to SD plates without Leu, Trp, and His. Plates were incubated at 30° C. and scored for growth of yeast and, hence, protein-protein interaction after two days.

The ETG1-MCM5 protein-protein interaction was confirmed in planta by BiFC experiments (Bracha-Drori et al., 2004; Walter et al., 2004). The full-length open reading frames of ETG1 and MCM5 with/without stop codon were amplified by PCR using F-ETG1 (5′-GGGGACAAGTTTGTACAAAAAAGCAGGCTTCATGGGAGGACCAGCTTACGATT-3′ (SEQ ID NO:24)) and R-ETG1 (5′-GGGGACCACTTTGTACAAGAAAGCTGGG TCTTACTTGAGCCTCTCCTTTCTA-3′ (SEQ ID NO:25)) primers, and F-MCM5 (5′-GGGGACAAGTTTGTACAAAAAAGCAGGCTTCATGTCAGGATGGGACGAAGGAG-3′ (SEQ ID NO:26)) and R-MCM5 (5′-GGGGACCACTTTGTACAAGAAAGCTG GGTCCTCAAGCTTTGCGGACAATAGAA-3′ (SEQ ID NO:27)) primers, respectively, and cloned into pDONR201 ENTRY vector by BP recombination reaction according to the manufacturer's instructions (Invitrogen).

Full-length open reading frames of ETG1 and MCM5 were transferred into the pH7FWG2 destination vector (Karimi et al., 2002) by LR recombination reaction, resulting in ETG1:EGFP and MCM5:EYFP fusion proteins, respectively. BiFC assay was performed as described by Walter et al. (2004). The coding region of ETG1 was amplified with 5′-GCCACTAGTGGATCCATGGGAGGACCAGCTTACGATT-3′ (SEQ ID NO:28) and 5′-AGCGGTACCCTCGAGGTACTTGAGCCTCTCCTTTCTA-3′ (SEQ ID NO:29) primers, and cloned via BamHI-XhoI into the plasmid pUC-SPYNE (Walter et al., 2004), resulting in the plasmid expressed as the ETG1:YFPN fusion protein; the coding region of MCM5 was also obtained with 5′-TGGCGCGCCACTAGTATGTCAGGATGGGACGAAGGAG-3′ (SEQ ID NO:30) and 5′-ACCCTCGAGGTCGACGTAAGCTTTGCGGACAATAGAA-3′ (SEQ ID NO:31) primers, and cloned via SpeI-SalI into the plasmid pUC-SPYCE (Walter et al., 2004) to give rise to the plasmid expressed as the MCM5:YFPC fusion protein.

The Agrobacterium strains containing both BiFC constructs were co-infiltrated into tobacco leaves. Epidermal cell layer of tobacco leaves were assayed for fluorescence using confocal laser scanning microscope (Zeiss) two to four days after infiltration. After transfection, YFP fluorescence was observed in the nuclei of cells transfected with the ETG1-YFPN and MCM5-YFPC fusion proteins, demonstrating that the ETG1 protein interacted with MCM5 in the plant nucleus (FIG. 5, Panel C; ETG1-YFPN and MCM5-YFPC). As expected, no fluorescence was detected when any combination with empty vectors was introduced into tobacco cells (FIG. 5, Panel C; ETG1-YFPN and YFPC, YFPN and MCM5-YFPC). When examining the subcellular localization of MCM5 in plants, with the use of an MCM5:eGFP fusion protein, MCM5:eGFP was found to reside in both the nucleus and cytoplasm (FIG. 5, Panel D).

To identify additional ETG1-associated proteins, tandem affinity purification (TAP) in combination with MALDI-TOF-TOF-MS based protein identification was performed. TAP experiments were done according to Van Leene et al. (2007). In short, the ETG1-coding sequence was cloned by recombination into the pKNTAP vector generating a Pro35S:TAP-ETG1 cassette (pKCETG1TAP). Arabidopsis cell suspension cultures were stably transformed by Agrobacterium-mediated co-cultivation with pKNETG1TAP. Transformed Arabidopsis cells were selected and transferred to liquid medium for upscaling. Expression levels of TAP-tagged proteins were checked by protein blotting with an anti-CBP antibody. In a first round of affinity purification, protein extracts of 15 g plant material were incubated with an IgG resin. Bound complexes were released and eluted from the resin by tag cleavage with TEV protease.

In a second affinity step on a calmodulin agarose column, co-eluting non-interacting proteins and the TEV protease were removed with the flow-through. Finally, both the ETG1 bait and interacting proteins were eluted from the calmodulin agarose via EGTA-mediated removal of calcium. Eluted proteins were separated on 4-12% NuPAGE gels, excised and analyzed by Maldi-TOF/TOF MS as described (Van Leene et al., 2007). To increase the stringency of the data set, contaminating proteins due to experimental background as determined by Van Leene et al. (2007) were systematically subtracted from the lists of co-purified proteins. Next to MCM5, we identified five interacting proteins, including other components of the MCM complex, being MCM2 (At1g44900), MCM3 (At5g46280), MCM4 (At2g16440), MCM6 (At5g44635) and MCM1 (At4g02060) (Table 2).

Combined with the subcellular localization results, these data indicated that ETG1 assembles into the replisome complex. Therefore, ETG1 depletion is expected to affect the efficiency of DNA replication. To test this hypothesis, a bromodeoxyuridine (BrdU) pulse-labeling experiment was performed. Three-day-old seedlings grown on MS agar plates were incubated in the labeling solution containing 10 μM bromodeoxyuridine (BrdU) (Roche) at room temperature for various time points, and the genomic DNA was extracted with a DNEASY® Plant Mini Kit (QIAGEN). The amounts of BrdU were determined by ELISA using Anti-BrdU-POD antibody (Roche). Three biological and two technical replicates were used at each time point for ELISA. Fifty microliters of the extracted DNA (0.2 μg/ml) was placed in each well. The ELISA procedure was chiefly that of 5-Bromo-2′-deoxy-uridine Labeling and Detection Kit III (Roche). In wild-type, the level of BrdU incorporation into cells gradually increased to a saturation level by three hours (FIG. 5, Panel E). By contrast, the rate of BrdU incorporation was lower in etg1-1 plants, illustrating the need for ETG1 for efficient DNA replication.

TABLE 1
Genes coexpressed with ETG1 gene.

| Rank | Cor. | Locus | Gene description |
|---|---|---|---|
| 1 | 0.92 | At5g46280 | Minichromosome maintenance family |
| | | | protein 3 (MCM3) |
| 2 | 0.90 | At1g44900 | Minichromosome maintenance family |
| | | | protein 2 (MCM2) |
| 3 | 0.89 | At4g02060 | Prolifera protein (PRL)/Minichromosome |
| | | | maintenance family protein 7 (MCM7) |
| 4 | 0.89 | At5g41880 | DNA primase small subunit |
| 5 | 0.88 | At1g07370 | Proliferating cell nuclear antigen 1 (PCNA1) |
| 6 | 0.88 | At2g16440 | Minichromosome maintenance family |
| | | | protein 4 (MCM4) |
| 7 | 0.87 | At2g07690 | Minichromosome maintenance family |
| | | | protein 5 (MCM5) |
| 8 | 0.84 | At1g67630 | DNA polymerase alpha subunit B |
| 9 | 0.84 | At5g67100 | DNA polymerase alpha catalytic subunit |
| 10 | 0.83 | At2g29570 | Proliferating cell nuclear antigen 2 (PCNA2) |

Top 10 ranking genes identified by ATTED-II coexpression database.
cor.: correlation coefficient

TABLE 2
List of ETG1-copurified proteins identified by Mass Spectrometry

| | | Protein | Peptide | Sequence | Protein score/ | Best ion score/ |
|---|---|---|---|---|---|---|
| Locus¹ | Gene description | MW | count | coverage % | threshold | threshold |
| At1g44900 | Minichromosome | 105172 | 33 | 43 | 936/61 | 104/33 |
| | maintenance | | | | | |
| | family protein 2 | | | | | |
| | (MCM2) | | | | | |
| At5g46280 | Minichromosome | 86759 | 14 | 23 | 206/61 | 74/31 |
| | maintenance | | | | | |
| | family protein 3 | | | | | |
| | (MCM3) | | | | | |
| At2g16440 | Minichromosome | 94168 | 24 | 38 | 696/61 | 102/28 |
| | maintenance | | | | | |
| | family protein 4 | | | | | |
| | (MCM4) | | | | | |
| At2g07690 | Minichromosome | 81591 | 26 | 38 | 584/61 | 107/31 |
| | maintenance | | | | | |
| | family protein 5 | | | | | |
| | (MCM5) | | | | | |
| At5g44635 | Minichromosome | 93478 | 20 | 33 | 859/61 | 154/30 |
| | maintenance | | | | | |
| | family protein 6 | | | | | |
| | (MCM6) | | | | | |
| At4g02060 | Prolifera protein | 80739 | 26 | 46 | 987/61 | 172/31 |
| | (PRL)/Minichromo- | | | | | |
| | some maintenance | | | | | |
| | family protein 7 | | | | | |
| | (MCM7) | | | | | |

¹All proteins were detected in the two independent TAP experiments.

### Example 6

### ETG1-Deficient Plants Activate the G2 DNA Replication Checkpoints

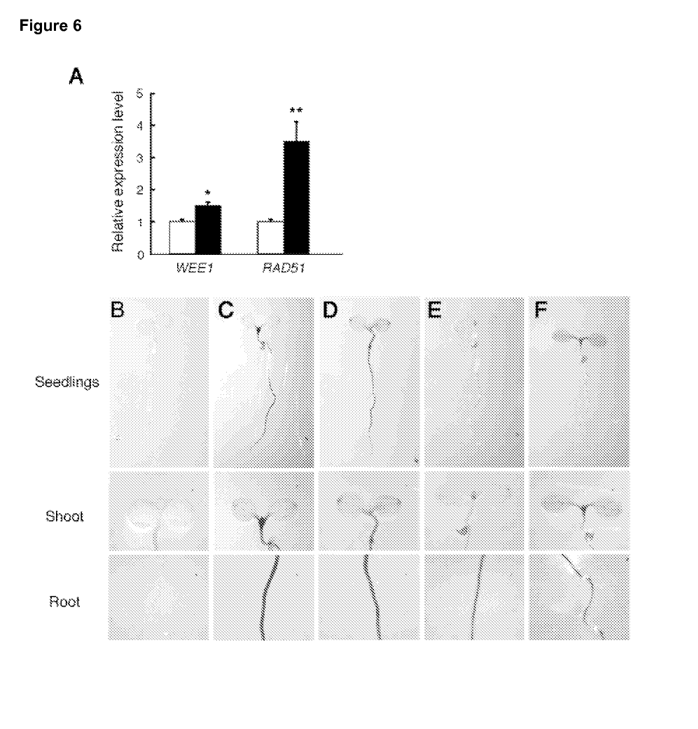

Inhibition of DNA replication in Arabidopsis results into the simultaneous induction of DNA repair genes and the cell cycle inhibitory WEE1 gene, which arrests cells in the G2 phase of the cell cycle (De Schutter et al., 2007). The decreased rate of DNA replication and observed interaction of ETG1 with replication proteins suggested that the G2-arrest noticed in ETG1-deficient plants might be the consequence of activation of the replication checkpoint. To test this hypothesis, we compared the expression levels of the RAD51 (DNA repair) and WEE1 (cell cycle checkpoint) marker genes by real-time RT-PCR in wild-type versus etg1 mutant plants. Ionizing radiation (γ-irradiation and UV) and radiomimetic drugs (HU, aphidicolin and bleomycin) are known to induce RAD51 and WEE1 expression (Chen et al., 2003; De Schutter et al., 2007). Expression of both RAD51 and WEE1 was significantly up-regulated in the etg1-1 seedlings (FIG. 6, Panel A). A similar expression profile was observed in etg1-2 seedlings.

Activation of the DNA stress checkpoint was confirmed by using plants that carried as transgene the poly(ADP-ribose) polymerase 2 (PARP2) and WEE1 promoter fused to GUS, being markers for DNA stress and activation of the G2 replication checkpoint, respectively (Babiychuk et al., 1998; Doucet-Chabeaud et al., 2001; De Schutter et al., 2007). As shown in FIG. 6, Panel B, no GUS activity was observed in PARP2::GUS plants grown under non-stress conditions. By contrast, treatment of the PARP2: GUS reporter line with bleomycin resulted a strong induction of GUS activity (FIG. 6, Panel D), demonstrating DNA stress-inducible promoter activity. Similarly, PARP2 promoter activity was induced in a etg1-1 background in the absence of any external DNA stress stimulus (FIG. 6, Panel C). Especially, GUS activity was strongly induced in shoot apical meristem and vascular cells. Analogous results were obtained using WEE1::GUS reporter plants. In control plants, WEE1 expression was observed in the shoot apex and vascular cells (FIG. 6, Panel E; De Schutter et al., 2007). This expression pattern was intensified in the etg1-1 background (FIG. 6, Panel F), confirming the real-time RT-PCR experiments.

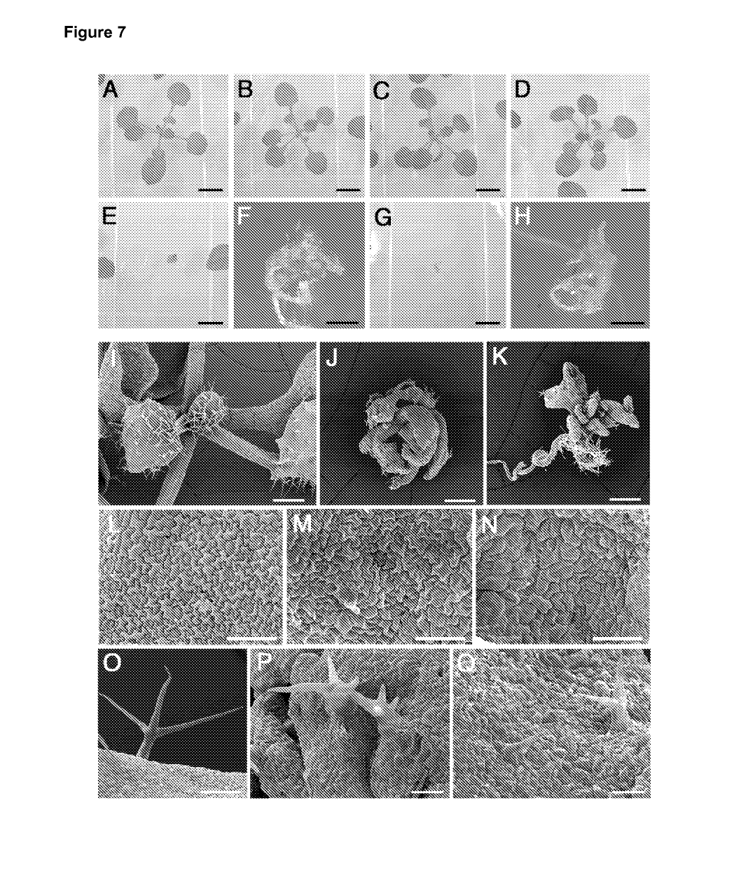

DNA replication stress caused by blocking of the replication fork is mainly sensed by the ATR kinase (Culligan et al., 2004). Previously, we have demonstrated that WEE1 is one of the main targets of the ATR signaling cascade. WEE1 transiently arrests cells in the G2 phase, allowing them to finalize DNA replication before proceeding into mitosis (De Schutter et al., 2007). When assuming that the increased cell cycle duration time observed in the etg1 mutant plants is the result of the activation of the replication checkpoint, it is expected that ETG1-deficiency should have a dramatic impact on the development of plants that are unable to arrest their cell cycle in response to DNA stress. To test this hypothesis, double mutants were constructed between etg1-1 and two DNA stress checkpoint mutants, atr-2 and wee1-1. The atr-2 and wee1-1 mutants have been described previously (Culligan et al., 2004; De Schutter et al., 2007). atr-2 and wee1-1 single mutants are hypersensitive to replication-blocking or DNA damaging drugs plants, but are viable and develop normally in the absence of exogenous DNA-stress treatments (FIG. 7, Panels A, C, and D) (Culligan et al., 2004; De Schutter et al., 2007). By contrast, etg1-1/wee1-1 and etg1-1/atr-2 double mutant plants showed a dwarf phenotype under non-stress conditions, illustrating a synthetic interaction between ETG1, and WEE1 or ATR (FIG. 7, Panels E-H).

Scanning electron microscopy revealed severe growth suppression (FIG. 7, Panels I-K). Especially, the size of trichomes was reduced in the double mutants (FIG. 7, Panels O-Q). No significant difference in leaf epidermal cell shape was observed in etg1-1/wee1-1 double mutants, whereas cells lost their jigsaw-like shape in etg1-1/atr-2 plants (FIG. 7, Panels L-N). The double mutants arrested at an early stage of development, indicating a cell cycle arrest. These results unequivocally illustrate that the activation of the DNA replication checkpoint in etg1 mutant plants is essential for their survival.

### Example 7

### Up-Regulation of Mitotic-Specific Genes in etg1

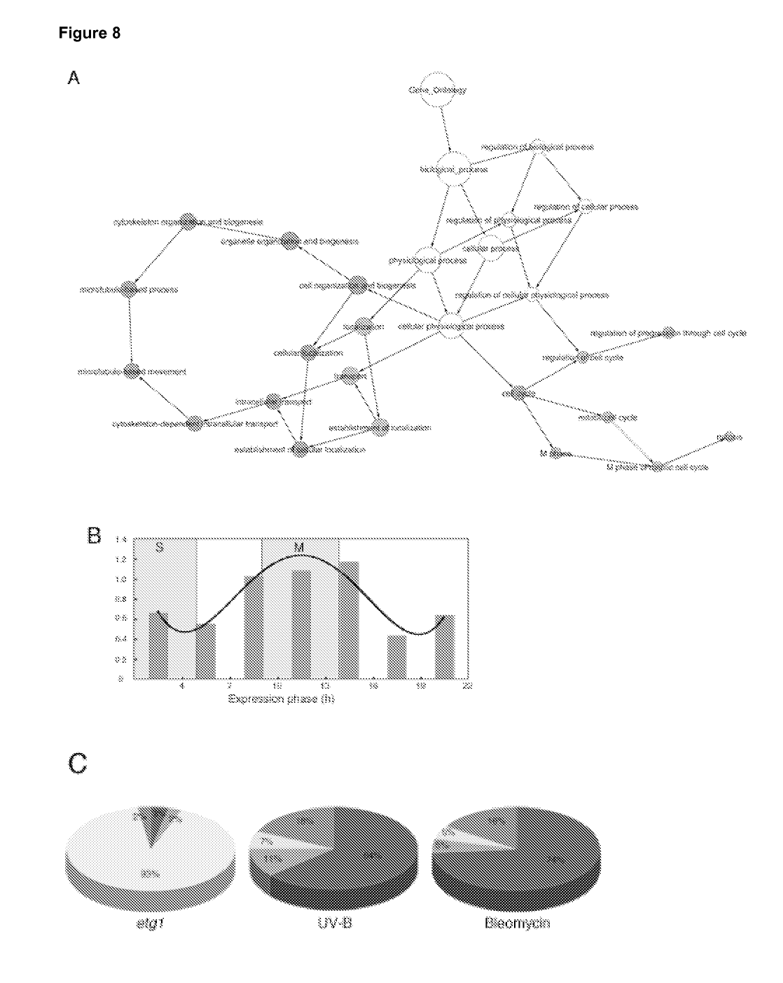

To gain more insight into cell cycle effect in etg1 mutants, we examined transcript levels of 24,000 genes by using Affymetrix ATH1 GeneChip arrays. Triplicate batches of first leaf pairs of nine-day-old wild-type and etg1 plants were harvested for total RNA preparation. The statistical analysis identified a total of 220 differentially expressed genes between wild-type and etg1 at P<0.01, among which 89% were up-regulated and 11% were down-regulated with fold change expression ranging from 1.3 to 14.8 and 0.15 to 0.75, respectively (Tables 3, 4). Interestingly, among the 196 up-regulated genes in etg1, 103 genes (52%) express with a peak in mitosis. Transcription of genes expressed specifically during mitosis is regulated by common upstream cis-acting element, called MSA (mitosis-specific activator; CAACGG). When we checked MSA element in promoter region of up-regulated genes in etg1, significant enrichment of MSA elements was detected. We also calculated the timing of maximal expression of up-regulated genes during the cell cycle. Genes expressed during M phase were clearly more strongly induced than those expressed during the S, G2, and G1 phases (FIG. 8, Panel B). To characterize biological processes, the up- and down-regulated genes were analyzed for gene ontology (G0) enrichment (Maere et al., 2005). Among the up-regulated genes in etg1, regulation of progression through cell cycle, mitotic cell cycle, and microtubule-based movement genes were significantly overrepresented (FIG. 8, Panel A), indicating that ETG1 is required for proper mitotic cell division. This is surprising, because ETG1 is expressed during S, and its gene product is required for DNA replication. Additionally, we have demonstrated in the previous examples that ETG1-deficient plants suffer from DNA stress, which is expected to result in a cell cycle arrest during S or early G2. We compared the microarray data of etg1 with gene expression changes observed in response to UV-B light and bleomycin treatment, both known to cause DNA breaks (Kilian et al., 2007; Molinier et al., 2005). Surprisingly, the response of cell cycle regulated genes was totally different in the etg1 mutant compared to the other treatments that cause DNA stress. In the latter cases, the set of modified genes was clearly enriched for S phase genes (FIG. 8, Panel C). These results suggest that in the etg1 mutant, different checkpoints pathways are activated to suppress cell cycle progression caused by DNA replication defects.

### Example 8

### ETG1 is Required for Sister Chromatid Cohesion

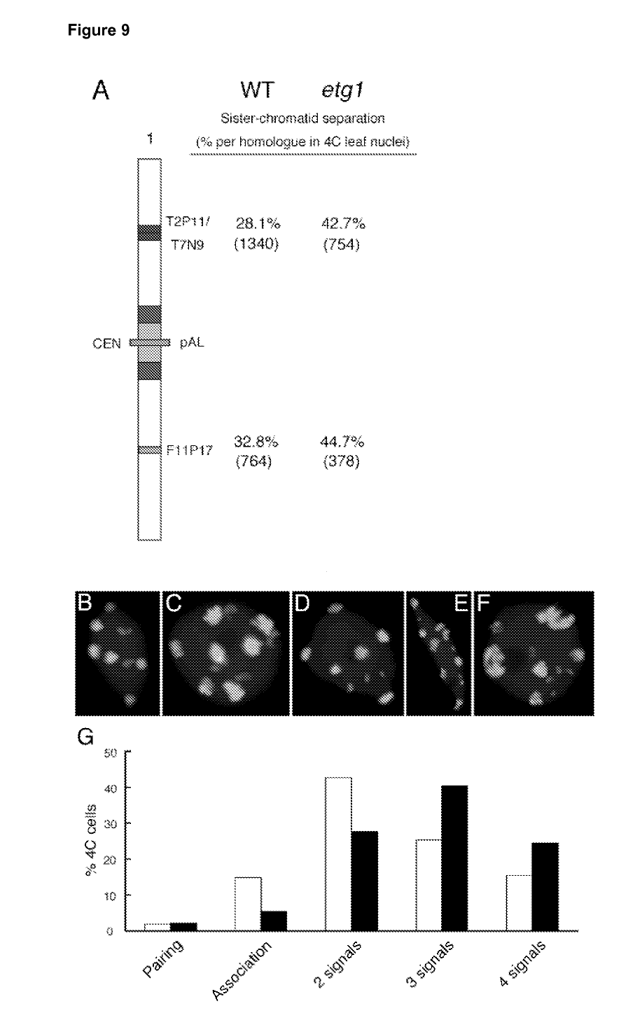

Sister chromatid cohesion is apparently established during DNA replication in S phase and maintained until anaphase onset. To assess the role of ETG1 in the establishment and maintenance of cohesion, we performed FISH analysis, as described by Schubert et al. (2008). To analyze sister chromatid cohesion in interphase nuclei, individual BACs (BAC T2P11/T7N9 and F11P17) from different positions along chromosomes 1 were hybridized to flow-sorted 4C leaf nuclei. The number of FISH signals was taken as a measure for sister chromatid separation. One FISH signal (FIG. 9, Panel B; pairing of both homologs) or two FISH signals (FIG. 9, Panels C and D) per BAC (T2P11/T7N9) were regarded as positional alignment at corresponding regions, indicating sister chromatids are aligned. Three (FIG. 9, Panel E) or four signals (FIG. 9, Panel F) were considered to indicate sister chromatid separation. Positional sister chromatid separation occurred in 28.1-32.8% of 4C nuclei in wild-type leaf, whereas etg1 leaf nuclei has 42.7-44.7% of sister chromatid separation (FIG. 9, Panel A). By comparing the DNA masses contained three or four FISH signals of wild-type and etg1 when using the BAC T2P11/T7N9, a significant increase in sister chromatid separation was observed in etg1 leaf nuclei (FIG. 9, Panel G). These findings reveal that ETG1 protein is required for establishment of sister chromatid cohesion.

TABLE 3
Up-regulated genes in etg1 compared with wild-type

| Fold change | | | | | |
|---|---|---|---|---|---|
| etg1-1 | etg1-2 | Gene code | Gene description | Phase | GO category |
| 6.91 | 5.92 | At5g60250 | Zinc finger (C3HC4-type ring | | Biological process unknown |
| | | | finger) family protein | | |
| 4.19 | 3.99 | At5g23910 | Kinesin motor protein-related | M | Microtubule-based |
| | | | | | movement |
| 3.51 | 3.55 | At4g37490 | CyclinB1; 1 (CycB1; 1) | | Regulation of progression |
| | | | | | through cell cycle |
| 3.39 | 3.15 | At4g02390 | Poly(ADP-ribose) polymerase | | Protein amino acid ADP- |
| | | | (APP) | | ribosylation |
| 3.35 | 3.20 | At5g61070 | Histone deacetylase 18 (HDA18) | | Chromatin remodeling |
| 3.32 | 3.39 | At3g44050 | Kinesin motor protein-related | M | Microtubule-based |
| | | | | | movement |
| 3.07 | 3.59 | At3g02120 | hydroxyproline-rich glycoprotein | M | Biological process unknown |
| | | | family protein | | |
| 3.05 | 3.25 | At5g45700 | NLI interacting factor (NIF) family | M | |
| | | | protein | | |
| 2.89 | 2.66 | At3g27060 | TSO2 | | DNA repair, DNA replication, |
| | | | | | regulation of progression |
| | | | | | through cell cycle |
| 2.80 | 2.56 | At3g23890 | TOPII (TOPOISOMERASE II) | M | DNA metabolic process |
| 2.79 | 2.65 | At5g51600 | PLE (PLEIADE) | M | Cytokinesis by cell plate |
| | | | | | formation |
| 2.78 | 2.95 | At4g35620 | CYCB2; 2 (Cyclin B2; 2) | M | Regulation of progression |
| | | | | | through cell cycle |
| 2.75 | 2.74 | At1g76540 | CDKB2; 1 (Cyclin-dependent | M | G2/M transition of mitotic cell |
| | | | kinase B2; 1) | | cycle |
| 2.68 | 2.59 | At3g51740 | IMK2 (Inflorescence meristem | M | Protein amino acid |
| | | | receptor-like kinase 2) | | phosphorylation |
| 2.68 | 2.47 | At5g55520 | Kinesin related protein | M | Biological process unknown |
| 2.66 | 2.94 | At3g22880 | ATDMC1 (RECA-LIKE GENE) | | Meiosis |
| 2.65 | 2.54 | At3g51280 | Male sterility MS5, putative | M | |
| 2.65 | 2.47 | At4g05520 | Calcium-binding EF hand family | M | |
| | | | protein | | |
| 2.61 | 2.58 | At5g56580 | ATMKK6 (Arabidopsis NQK1) | | Protein amino acid |
| | | | | | phosphorylation |
| 2.60 | 2.47 | At2g25880 | ATAUR2 (ATAURORA2) | M | Histone phosphorylation |
| 2.56 | 2.71 | At2g27970 | CKS2 (CDK-subunit 2) | | Cell cycle |
| 2.55 | 2.66 | At4g05190 | ATK5 (Arabidopsis thaliana | M | Microtubule cytoskeleton |
| | | | kinesin 5) | | organization and biogenesis, |
| | | | | | spindle assembly |
| 2.53 | 2.76 | At2g30360 | CIPK11 | | Protein amino acid |
| | | | | | phosphorylation, signal |
| | | | | | transduction |
| 2.53 | 2.57 | At5g11510 | MYB3R-4 (c-myb-like transcription | M | Cell cycle, regulation of |
| | | | factor 3R-4) | | transcription |
| 2.52 | 2.49 | At4g01730 | Zinc finger (DHHC type) family | M | Biological process unknown |
| | | | protein | | |
| 2.51 | 2.51 | At1g76310 | CYCB2; 4 (CYCLIN B2; 4) | M | Regulation of progression |
| | | | | | through cell cycle |
| 2.51 | 2.87 | At5g55180 | Glycosyl hydrolase family 17 | M | Carbohydrate metabolic |
| | | | protein | | process |
| 2.47 | 2.60 | At1g08560 | KN (KNOLLE) | M | Intracellular protein transport |
| 2.47 | 2.58 | At4g32830 | ATAUR1 (ATAURORA1) | M | Histone phosphorylation |
| 2.46 | 2.58 | At1g20930 | CDKB2; 2 (CYCLIN-DEPENDENT | M | M phase of mitotic cell cycle |
| | | | KINASE B2; 2) | | |
| 2.44 | 2.61 | At2g26760 | CYCB1; 4 | M | Regulation of progression |
| | | | | | through cell cycle |
| 2.44 | 2.30 | At5g60930 | Chromosome-associated kinesin | M | Microtubule-based |
| | | | | | movement |
| 2.43 | 2.57 | At1g18370 | HINKEL (HIK) | M | Microtubule-based |
| | | | | | movement |
| 2.43 | 2.74 | At2g37420 | Kinesin motor protein-related | M | Microtubule-based |
| | | | | | movement |
| 2.41 | 2.71 | At2g17620 | CYCB2; 1 (CYCLIN B2; 1) | M | Regulation of progression |
| | | | | | through cell cycle |
| 2.37 | 2.60 | At3g25980 | Mitotic spindle checkpoint protein, | M | Mitotic cell cycle spindle |
| | | | putative (MAD2) | | assembly checkpoint |
| 2.36 | 2.29 | At3g23670 | PAKRP1L | M | Microtubule-based |
| | | | | | movement |
| 2.36 | 2.46 | At1g28290 | Pollen Ole e 1 allergen and | M | Biological process unknown |
| | | | extensin family protein | | |
| 2.35 | 2.32 | At1g44110 | CYCA1; 1 (CYCLIN A1; 1); | M | Regulation of progression |
| | | | | | through cell cycle |
| 2.33 | 2.22 | At3g06030 | Arabidopsis NPK1-related protein | | Protein amino acid |
| | | | kinase 3 (ANP3) | | phosphorylation |
| 2.32 | 1.89 | At3g60840 | Microtubule associated protein | M | |
| | | | (MAP65/ASE1) family protein | | |
| 2.30 | 2.53 | At3g20150 | Kinesin motor family protein | M | Microtubule-based |
| | | | | | movement |
| 2.29 | 2.34 | At5g13840 | ccs52B | M | Signal transduction |
| 2.29 | 2.71 | At1g34355 | Forkhead-associated domain- | M | Biological process unknown |
| | | | containing protein | | |
| 2.27 | 2.10 | At1g02690 | Importin alpha-2 subunit, putative | M | Intracellular protein transport |
| 2.28 | 2.29 | At1g02730 | Cellulose synthase-like D5 | M | cellulose synthase activity |
| | | | (ATCSLD5) | | |
| 2.28 | 2.30 | At5g66230 | Similar to sugar transporter | M | Biological process unknown |
| | | | superfamily | | |
| 2.26 | 2.23 | At1g50240 | FUSED (FU) | | Cellularization of the embryo |
| | | | | | sac, cytokinesis by cell plate |
| | | | | | formation |
| 2.26 | 2.00 | At1g63100 | SCARECROW transcription factor | M | Regulation of transcription |
| 2.26 | 2.18 | At1g18250 | Arabidopsis thaumatin-like protein | M | Response to other organism |
| | | | 1 (ATLP-1) | | |
| 2.25 | 2.08 | At1g72250 | Kinesin motor protein-related | M | Microtubule-based |
| | | | | | movement |
| 2.24 | 2.13 | At4g31805 | WRKY family transcription factor | | Regulation of transcription |
| 2.24 | 2.04 | At1g03780 | Targeting protein-related | M | Biological process unknown |
| 2.23 | 2.40 | At5g67270 | Microtuble-end-binding protein 1 | M | Cortical cytoskeleton |
| | | | (ATEB1C) | | organization and biogenesis |
| 2.23 | 2.91 | At2g18600 | RUB1-conjugating enzyme, | | Protein modification process, |
| | | | putative | | ubiquitin cycle |
| 2.21 | 1.99 | At3g57860 | UVB-insensitive 4-like | | Biological process unknown |
| 2.12 | 2.06 | At1g50490 | Ubiquitin-conjugating enzyme 20 | M | Cell proliferation |
| | | | (UBC20) | | |
| 2.12 | 1.87 | At3g17360 | Phragmoplast orienting kinesin 1 | M | Microtubule-based |
| | | | (POK1) | | movement |
| 2.10 | 2.32 | At5g47500 | Pectinesterase family protein | M | Cell wall modification |
| 2.10 | 2.14 | At2g28620 | Kinesin motor protein-related | M | Microtubule-based |
| | | | | | movement |
| 2.09 | 1.96 | At2g22610 | Kinesin motor protein-related | M | Microtubule-based |
| | | | | | movement |
| 2.07 | 2.03 | At4g21820 | Calmodulin-binding family protein | M | |
| 2.07 | 1.98 | At1g66620 | Seven in absentia (SINA) protein, | | Multicellular organismal |
| | | | putative | | development, ubiquitin- |
| | | | | | dependent protein catabolic |
| | | | | | process |
| 2.07 | 1.90 | At1g23000 | Heavy-metal-associated domain- | | Metal ion transport |
| | | | containing protein | | |
| 2.07 | 2.33 | At4g11080 | High mobility group (HMG1/2) | M | Regulation of transcription |
| | | | family protein | | |
| 2.05 | 1.95 | At1g73620 | Thaumatin-like protein, putative | | Response to other organism |
| 2.03 | 2.21 | At1g78430 | Tropomyosin-related | | Biological process unknown |
| 2.03 | 1.87 | At2g33560 | Spindle checkpoint protein-related | M | Biological process unknown |
| 2.02 | 2.05 | At3g55660 | ATROPGEF6/ROPGEF6 (Kinase | M | Biological process unknown |
| | | | partner protein-like) | | |
| 2.00 | 1.79 | At4g14330 | Phragmoplast-associated kinesin- | M | Microtubule-based |
| | | | related protein 2 (PAKRP2) | | movement |
| 1.99 | 1.78 | At5g40840 | Sister chromatid cohesion 1 | | Mitosis |
| | | | (SCC1) protein homolog 2 (SYN2) | | |
| 1.98 | 1.74 | At5g27550 | Kinesin motor protein-related | M | Microtubule-based |
| | | | | | movement |
| 1.96 | 2.04 | At1g10780 | F-box family protein | M | Biological process unknown |
| 1.94 | 2.16 | At1g59540 | Kinesin-like protein | M | Microtubule-based |
| | | | | | movement |
| 1.92 | 1.99 | At1g53140 | Dynamin family protein | M | Biological process unknown |
| 1.92 | 1.67 | At3g10310 | Kinesin motor protein-related | M | Microtubule-based |
| | | | | | movement |
| 1.91 | 1.77 | At5g03780 | TRF-like 10 (TRFL10) | | Response to salicylic acid |
| | | | | | stimulus |
| 1.90 | 1.69 | At2g36200 | Kinesin motor protein-related | | Microtubule-based |
| | | | | | movement |
| 1.90 | 1.65 | At1g57820 | Variant in methylation 1 (VIM1) | | Regulation of transcription, |
| | | | | | DNA-dependent, centric |
| | | | | | heterochromatin formation, |
| | | | | | DNA methylation on cytosine |
| 1.89 | 1.83 | At4g03100 | Rac GTPase activating protein, | G2 | Signal transduction |
| | | | putative | | |
| 1.87 | 1.79 | At3g27330 | Zinc finger (C3HC4-type RING | M | Protein ubiquitination |
| | | | finger) family protein | | |
| 1.87 | 1.90 | At4g33400 | Defective embryo and meristems | M | N-terminal protein |
| | | | protein-related (DEM) | | myristoylation |
| 1.86 | 1.98 | At4g21270 | ATK1 (Arabidopsis thaliana | | Anastral spindle assembly |
| | | | kinesin 1) | | involved in male meiosis |
| 1.85 | 1.83 | At4g28950 | ARAC7/ATROP9/RAC7/ROP9 | S | Small GTPase mediated |
| | | | | | signal transduction |
| 1.85 | 2.08 | At2g25060 | Plastocyanin-like domain- | M | Electron transport |
| | | | containing protein | | |
| 1.84 | 1.97 | At4g31840 | Plastocyanin-like domain- | M | Electron transport |
| | | | containing protein | | |
| 1.84 | 1.87 | At5g03870 | Glutaredoxin family protein | M | N-terminal protein |
| | | | | | myristoylation |
| 1.84 | 2.06 | At2g07170 | Similar to TORTIFOLIA 1 (TOR1) | M | Biological process unknown |
| 1.82 | 1.73 | At5g63920 | DNA topoisomerase III alpha, | | DNA topological change, |
| | | | putative | | DNA unwinding during |
| | | | | | replication |
| 1.80 | 2.15 | At5g55830 | Lectin protein kinase, putative | M | Protein amino acid |
| | | | | | phosphorylation |

### Example 9

### Constitutive Knock-Down and Overexpression of hETG1 Causes Substantial Changes in Cellular Morphology

The human ETG1 cDNA (hETG1) was PCR amplified from the 2961492 clone (openbiosystem) using PFU turbo (Stratagene) and the following primers 5′-CACCATGCCGTGTGGGGAGG-3 (SEQ ID NO:32) and 5′-TCTAGAAAGTTCATTTCCATTCACACATTT-3′ (SEQ ID NO:33); following the manufacturer's instruction. The PCR product was ligated in the pENTR/D/Topo vector (InVitrogen) according to the manufacturer instruction.

To study the role of human hETG1, transgenic MCF7 cell cultures were generated that either overexpress or silence the hETG1 gene. MCF-7 cell lines were obtained from the American Cell Type Culture Collection (Rockville, Md.) and maintained in DMEM supplemented with 5% FCS, 5% newborn bovine serum, 2 mmol/L-glutamine, 0.4 mmol/L sodium pyruvate, 100 units/mL penicillin and 100 μg/mL streptomycin and in DMEM supplemented with 5% FCS, 2 mmol/L L-glutamine, 0.4 mmol/L sodium pyruvate, 100 units/mL penicillin, 100 μg/mL streptomycin, 6 ng/ml bovine insulin (Sigma-Aldrich, St Louis, Mo.), respectively. All recombinant lentiviruses were produced by transient transfection of HEK293T cells according to standard protocols. Briefly, 0.6 million cells of the packaging cell line HEK293T were seeded in two wells of six-well plates. After 24 hours, 3 μg of the lentiviral knock-down vector pGIZ V2HS-158067 purchased from Open-Biosystems (Huntsville, Ala.), 3 μg of the packaging plasmid pCMV-ΔR8.91 (Zufferey et al, 1997), and 1.5 μg of the envelope plasmid pMD2G-VSVG (Zufferey et al, 1997) were first ethanol-precipitated together and then transfected in the presence of chloroquine (25 μM) into the HEK293T cells using the calcium phosphate precipitation method.

Transduction of the MCF7 cells was performed in triplicate by resuspending 25,000 cells with 200 ml viral supernatant and plating them in a 96-well plate. The plate was centrifuged for 1.5 hours at 32° C. and 1500 rpm and incubated at 37° C. in a water-saturated incubator under a 5% O₂/95% CO₂ atmosphere. After 96 hours, the cells were trypsinized, pooled and amplified. Transduction efficiencies were determined by measuring EGFP expression using FACS analysis (Epics Altra from Beckman Coulter, Fullerton, Calif., USA). For the overproduction of hETG, we modified the pWPI (addgene ref 12254) lentiviral vector (Pham et al., 2004) to include a C-terminal tag (Myc) at the end of the gene of interest, a Tet operon sequence in front of the promoter sequence of the vector to allow conditional control of the expression cassette and finally a Gateway® cloning cassette located between the promoter and a C-terminal tag (Myc or V5His) to allow rapid transfer of the genes of interest from Gateway-compatible entry vectors. Co-transduction with a suitable lentiviral vector, pLV-tTR-KRAB-Red (Wiznerowicz and Trono, 2003), allows controlling the expression of the transgene by addition of doxycycline. Finally, the vector also bears an EGFP selection marker driven by an IRES sequence following the Gateway cassette to follow infection efficiency and eventually enrich the population by FACS sorting.

Overexpression and silencing of the hETG1 gene were confirmed by RT-PCR analysis. RNA was extracted from MCF-7 cells with the RNeasy Plus Mini Kit (Qiagen, Hilden, Germany). cDNA was prepared from 1 μg total RNA with the iScript cDNA synthesis kit (Bio-Rad, Hercules, Calif.) according to the manufacturer's instructions. For quantitative PCR, PCR reactions were run in triplicate on a LightCycler® 480 Real-Time PCR system (Roche) using the SYBR Green I master Mix (Roche), 100 nM primers and 20 ng of cDNA according to the manufacturer's instructions The CT (threshold cycle when fluorescence intensity exceeds ten times the SD of the baseline fluorescence) values for the target amplicon and endogenous control (TBP) were determined for each sample. Quantification was performed using the comparative C_{T} method (DDC_{T}) Primers used were 5′-ACTCTCCACGAAATACCACTTTG-3′ (SEQ ID NO:34) and 5′-GTAGGATGTTGAGGGACTGACTCG-3′ (SEQ ID NO:35) for hETGJ and 5′-CGGCTGTTTAACTTCGCTTC-3′ (SEQ ID NO:36) and 5′-CACACGCCAAGAAACAGTGA-3′ (SEQ ID NO:37) for TBP. For both the overexpressing and silencing lines appropriate cell lines were selected. The V5-C10 overexpressing line showed an 17-fold induction of hETG1 expression levels. The 158067-Const knock-down cell line showed a depletion of 80% in transcription of hETG1.

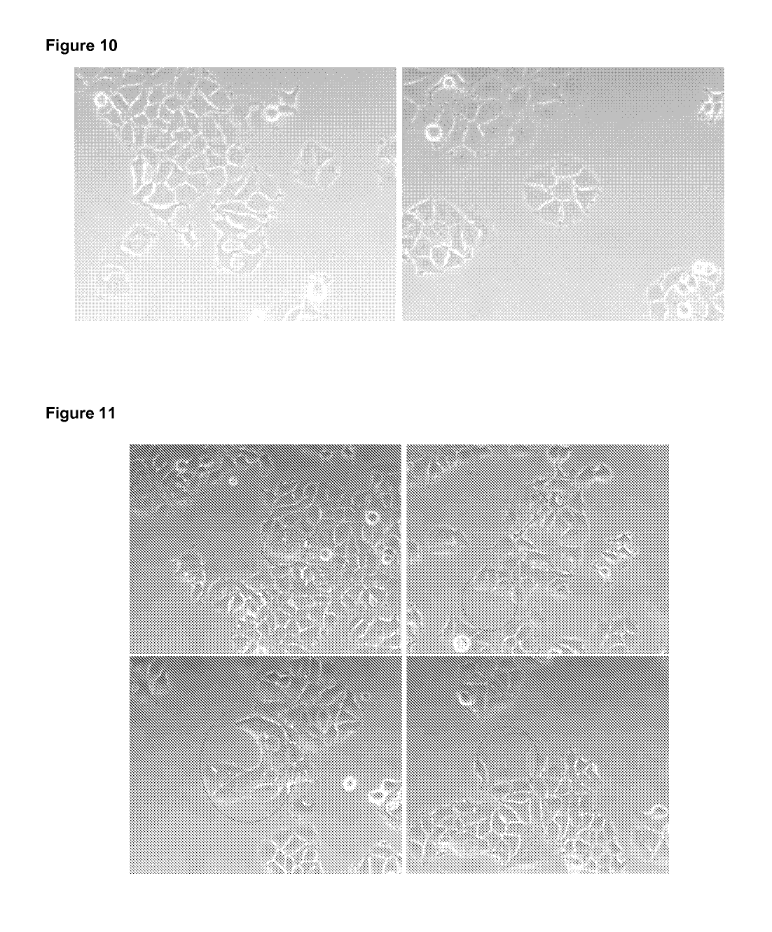

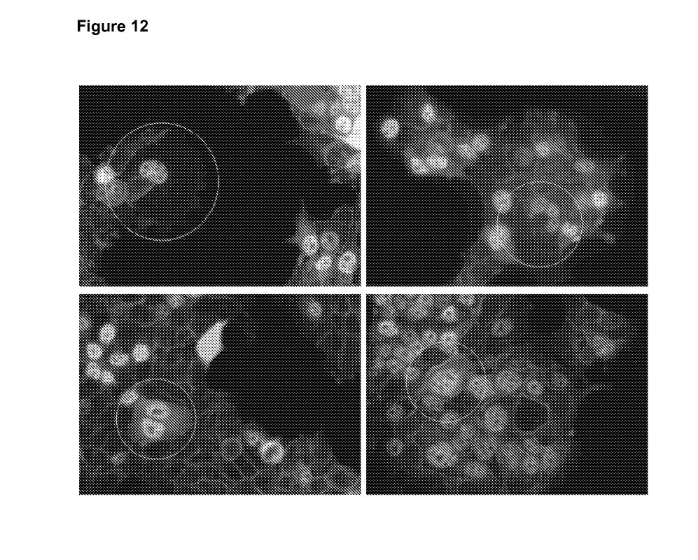

In both the hETG1 overexpression and knock-down lines substantial changes in the cellular morphology were observed. These changes were characterized by the appearance of multi-nucleated and/or giant cells (FIGS. 10 and 11). To assess more clearly the severity of the phenotype observed after hETG1 over-expression or depletion, co-stainings of hETG1 with the membrane marker β-Catenin were performed. Twenty thousand MCF-7 cells were grown for two days at 37° C. Cells were then washed with 1 ml of PBS three times and fixed with 500 μl of ice-cold 100% methanol. Fixed cells were first incubated 1 hour at room temperature in 200 PBS supplemented with 0.04% gelatin, then for 1 hour with primary antibodies (Anti-V5-His-Tag antibody, Santa Cruz Biotechnology INC, and Anti-β-Catenin antibody, Santa Cruz Biotechnology INC) diluted in PBS. Cells were next washed three times with PBS and incubated for 1 hour at room temperature with secondary antibody (Alexa-488 anti-mouse, Ig, 1:5000 dilution; Invitrogen, Carlsbad, Calif. for V5-His Tag and Alexa-594 anti-rat, Ig, 1:5000 dilution; Invitrogen, Carlsbad, Calif. for (3-Catenin). After three washes with PBS, coverslips were mounted on microscopic glass slides using vecatshield supplemented with DAPI (Vector Laboratories, Burlingame, Calif.) to prevent photobleaching. Coverslips were examined with an Olympus BX61 fluorescent microscope under a magnification of 40×. Examples of giant multinucleated cells can be observed in FIG. 12. These data indicate a clear cytokinesis defect upon modulation of the hETG1 expression level.

### Example 10

### Constitutive Knock-Down of hETG1 Causes a G2 Cell Cycle Arrest

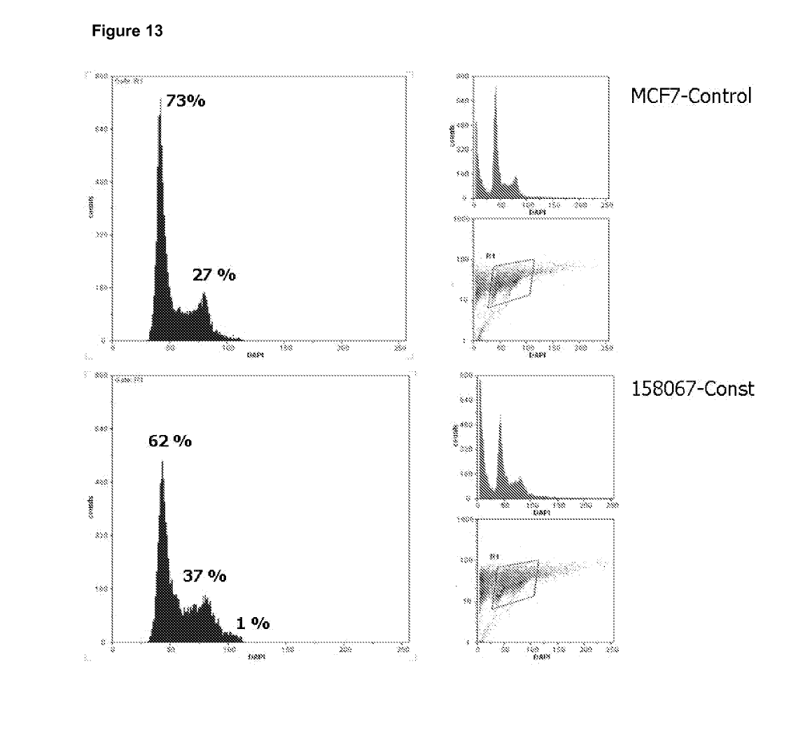

Two hundred thousand hETG1 knocked-down and control MCF-7 cells (non infected MCF-7) were plated into six-well plates and grown two days in 4 ml of MCF-7 medium. Cells were next trypsinized, centrifuged at 2000 rpm for 5 minutes re-suspended in 1 ml of FAC's buffer (PBS pH 7.2, 0.5% BSA and 2 mM EDTA) and filtered on a 40 μm strainer (Becton Dickinson, San Jose, Calif.). Cells were incubated on ice in the dark during 15 minutes in FACS buffer supplemented with DAPI. The DNA content was analyzed in triplicate by flow cytometry. Compared to the control cultures, the knocked-down cultures are characterized by a depletion of the 2C (G1-phase) cell population, correlated with an increment in the population of cells with a DNA content equal to 4C (G2-phase) or greater (polyploidy) (FIG. 13), indicating an arrest in their G2 cell cycle phase.

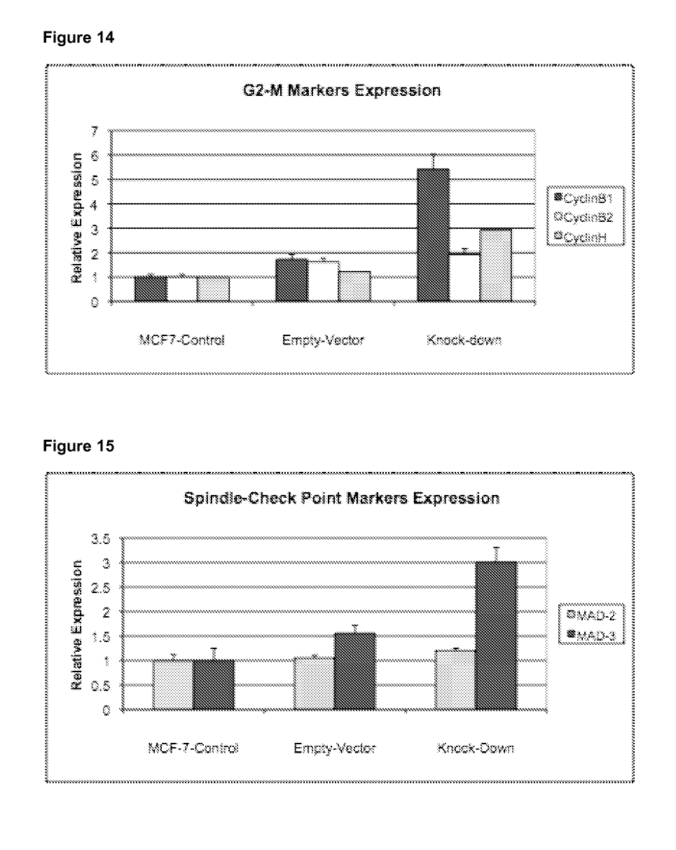

This cell cycle arrest was further demonstrated by transcriptional up-regulation of G2-M marker genes (such as Cyclin B1, Cyclin B2 and Cyclin H) (FIG. 14), illustrating the importance of functional h-ETG1 for normal cell cycle progression. The combined appearance a G2-M arrest and multi-nucleated cell phenotype indicated defects during sister chromatid organization and/or separation. Recent work has shown that the spindle checkpoint inhibits cell-cycle progression by direct binding of components of the spindle checkpoint pathway or kinetochore-associated proteins (like Mad2 and/or Mad3) to components of the anaphase-promoting complex, resulting into an M-phase arrest (Malureanu et al. 2009; Kulukian et al. 2009). Knocked-down h-ETG1 cells display a clear up-regulation of Mad2 and Mad3 transcripts, corroborating the view that specific problems during sister chromatid separation are at least partly responsible for the observed phenotypes (FIG. 15).

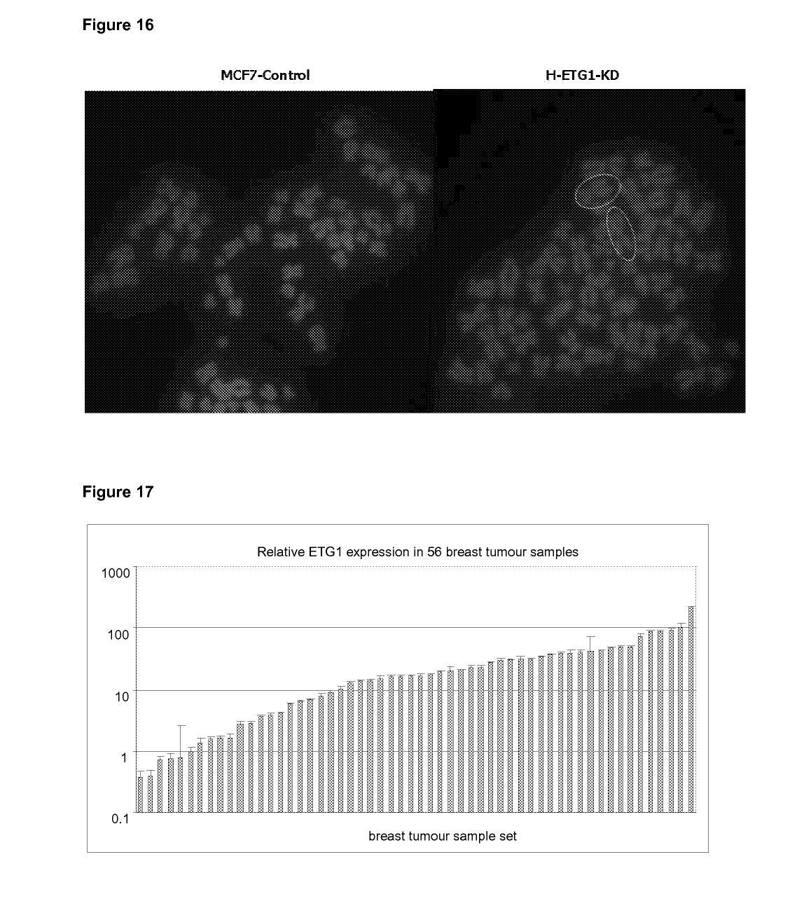

To further investigate this issue, metaphases in MCF7 wild-type and h-ETG1 knock-down karyotypes were characterized by counting metaphase chromosomes with totally detached chromatids. Briefly, upon h-ETG1 knock-down, cells were cultivated during 2 days at 37° C. To enrich for mitotic chromosomes, subconfluent cells were treated with KaryoMax colcemid (Sigma) for five hours before harvesting. Cells were trypsinized, pelleted and resuspended in hypotonic solution (60 mM KCl) for 30 minutes at room temperature. Cells were repelleted, the hypotonic solution was removed and cells were resuspended in freshly made methanol: glacial acetic acid (3:1) added drop-wise. Two or three drops of suspended cells were applied to precleaned blood smear glass slides and finally chromosomes were counterstained with VectaShield containing DAPI (Vector Laboratories). Microscopic analysis illustrated an increment of chromosomes with totally detached metaphase chromosomes upon h-ETG1 knock-down (FIG. 16), clearly indicating that h-ETG1 plays a fundamental role during chromosome segregation, and that reduced sister chromatids cohesion is a main reason for the observed multi-nucleated cell phenotype.

### Example 11

### ETG1 Expression Analysis in Human Primary Breast Cancer

cDNA synthesis on RNA samples was performed on 1.5 μg total RNA using the Iscript cDNA synthesis kit (Bio-Rad). Subsequently qPCR on the LC480 (Roche) was done for ETG1 and different reference genes (Vandesompele et al. 2002) using LCS480 Sybr Green I master kit (Roche), Fast SYBR master mix kit (Applied Biosystems) and Taqman fast univ. PCR Mastermix (Applied Biosystems). Using GeNorm (Vandesompele et al. 2002) we determined the most accurate set of reference genes for normalisation (HMBS, ACTB, HPRTI, RPL13A, SDHA, TBP and UBC). The average threshold cycle of triplicate reactions was used for all subsequent calculations using the delta Ct method. Relative ETG1 expression levels (average of ten samples with low expression set to 1) were depicted ranking low to high (FIG. 17).

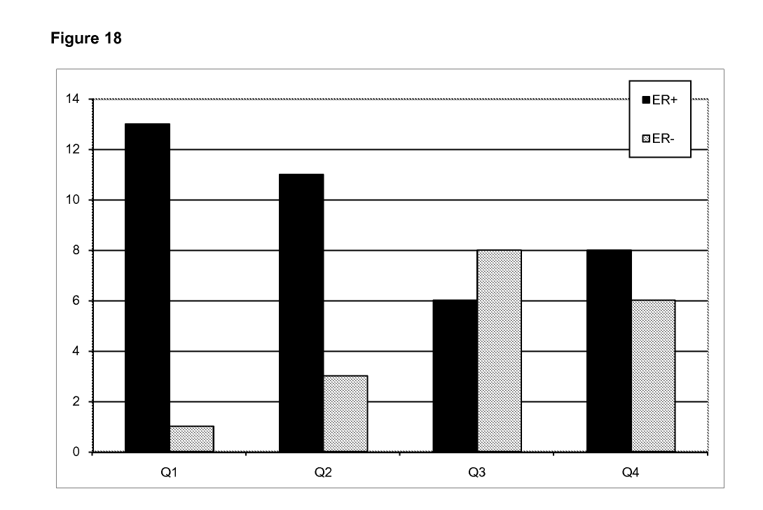

The expression of ETG1 was further compared with different clinicopathological parameters available for the different analyzed tumors. High ETG1 expression was correlated with a negative estrogen receptor (ER) status (FIG. 18). For breast cancers, mRNA expression profiling has shown that one of the most powerful denominators in determining the gene expression signatures and prognostic groups of breast cancer is estrogen receptor (ER) and ER-related genes. Breast cancers have been separated by gene expression profiles into luminal, basal like, ERBB2, and normal breast-like subgroups (Sorlie et al., 2001). Basal-like tumors express many of the genes characteristic of breast basal epithelial cells and the most typical feature of basal like breast cancers is the lack of expression of ER and genes usually co-expressed with ER (Perou et al., 2000). This negative ER status is a well established prognostic and predictive marker in breast cancer. Microarray studies have shown that basal-like tumors have poor prognosis when compared with ER-positive luminal tumor groups (Sorlie et al., 2003). This finding supports the importance and usefulness of assessing the protein status of ETG1 in human cancer samples.

### Example 12

### ETG1 as a Pre-Cancer Marker

Early detection of cancer by screening remains an important effective method for improving cancer survival. ETG1 is tested for potential clinical practice by the generation of diagnostic antibodies. A full-length human ETG1 cDNA clone was used as a template for PCR amplification with Pfu polymerase to generate an ETG1 coding cassette. The primers used for amplification were hETG1-Inf-Fw 5′-CAAGGTACCAAGCTTAATGCCGTGTGGGGAGG-3′ (SEQ ID NO:38) and hETG1-Inf-Rv 5′-TGCGGCCGCATGCATTTAAAGTTCATTTCCAT-3′ (SEQ ID NO:39). The resulting PCR product was inserted by fusion cloning into the HindIII pLHX32 plasmid downstream of the His₆ tag, to generate pLHXhETG1. Plasmid insert is controlled by DNA sequencing. Plasmid is transformed in MC 1061 bacteria containing a pICA2 plasmid. Exponentially growing E. Coli bacteria are induced overnight with 1 mM isopropyl β-D-thiogalactopyranoside at 20° C. The cells are harvested by centrifugation and the cell paste is frozen until required. Frozen MC1061 cell pellets are suspended in buffer A, comprising 20 nM Tris-HCl, pH 7.5, 10% glycerol, 1 mM oxidized glutathione, 200 mM NaCl, 1 mM phenylmethylsulfonyl fluoride, 50 μM leupeptin and 20 μg/ml aprotinin, and are lysed by sonication or French press. Insoluble proteins are removed by centrifugation. Bacterial DNA is removed over a DEAF column equilibrated by buffer A. The flow through is applied on a Co⁺² metal chelate column which is washed with buffer A for 4 to 16 hours. Low strength metal binding proteins are removed by a short washing with buffer B, consisting of 20 mM Tris-HCl pH 7.5, 10% glycerol, 1 mM oxidized glutathione, 200 mM NaCl and 10 mM imidazole. His₆-tagged ETG1 is eluted from the column by buffer C, containing 20 mM Tris-HCl pH 7.5, 10% glycerol, 1 mM oxidized glutathione, 50 mM NaCl and 100 mM imidazole. The purity of the preparation is checked by SDS-PAGE and further purified if needed by Q-resource or monoQ columns. Purified proteins are used for immunization of rabbits for the production of polyclonal antibodies (Eurogentec). In addition purified ETG1 protein is provided to the VIB Nano-body service facility for lama immunization. Generated anti-ETG1 polyclonal antibodies and nanobodies are used for a wide range of applications like immunohistochemistry, immunomodulation, intracellular expression (intrabodies), biosensor-applications, etc. Collaborations are initiated with different pathology departments from which Tissue MicroArrays (TMAs) are utilized for high throughput molecular pathology characterization of various kinds of tumors (e.g. skin, colon, breast). TMAs containing samples with long-term and complete clinical follow-up data are available within this framework, allowing us to obtain prognostic and predictive information.

### REFERENCES

Aparicio, O. M., Weinstein, D. M. and Bell, S. P. (1997). Components and dynamics of DNA replication complexes in S. cerevisiae: redistribution of MCM proteins and Cdc45p during S phase. Cell 91: 59-69.
Babiychuk, E., Cottrill, P. B., Storozhenko, S., Fuangthong, M., Chen, Y., O'Farrell, M. K., Van Montagu, M., Inzé, D. and Kushnir, S. (1998). Higher plants possess two structurally different poly(ADPribose) polymerases. Plant J. 15: 635-645.
Beeckman, T. and Engler, G. (1994). An easy technique for the clearing of histochemically stained plant tissue. Plant Mol. Biol. Rep. 12: 37-42.
Bell, S. P. and Dutta, A. (2002). DNA replication in eukaryotic cells. Annu. Rev. Biochem. 71: 333-374.
Boudolf, V., Vlieghe, K., Beemster, G. T., Magyar, Z., Torres Acosta, J. A., Maes, S., Van Der Schueren, E., Inzé, D. and De Veylder, L. (2004). The plant-specific cyclin-dependent kinase CDKB1;1 and transcription factor E2Fa-DPa control the balance of mitotically dividing and endoreduplicating cells in Arabidopsis. Plant Cell 16: 2683-2692.
Bowler, C., Benvenuto, G., Laflamme, P., Molino, D., Probst, A. V., Tariq, M., Paszkowski, J. (2004). Chromatin techniques for plant cells. Plant J. 39: 776-789.
Bracha-Drori, K., Shichrur, K., Katz, A., Oliva, M., Angelovici, R., Yalovsky, S, and Ohad, N. (2004). Detection of protein-protein interactions in plants using bimolecular fluorescence complementation. Plant J. 40: 419-427
Chen, I. P., Haehnel, U., Altschmied, L., Schubert, I. and Puchta, H. (2003). The transcriptional response of Arabidopsis to genotoxic stress—a high-density colony array study (HDCA). Plant J. 35: 771-786.
Clough, S. J. and Bent, A. F. (1998). Floral dip: A simplified method for Agrobacterium-mediated transformation of Arabidopsis thaliana. Plant J. 16: 735-743.
Culligan, K., Tissier, A. and Britt, A. (2004). ATR regulates a G2-phase cell-cycle checkpoint in Arabidopsis thaliana. Plant Cell 16: 1091-1104.
De Schutter, K., Joubès, J., Cools, T., Verkest, A., Corellou, F., Babiychuk, E., Van Der Schueren, E., Beeckman, T., Kushnir, S., Inzé, D. and De Veylder, L. (2007) Arabidopsis WEE1 kinase controls cell cycle arrest in response to activation of the DNA integrity checkpoint. Plant Cell 19: 211-25.
De Veylder, L., Beeckman, T., Beemster, G. T., Krols, L., Terras, F., Landrieu, I., van der Schueren, E., Maes, S., Naudts, M. and Inze, D. (2001). Functional analysis of cyclin-dependent kinase inhibitors of Arabidopsis. Plant Cell 13: 1653-1668.
De Veylder, L., Beeckman, T., Beemster, G. T. S., de Almeida Engler, J., Ormenese, S., Maes, S., Naudts, M., Van Der Schueren, E., Jacqmard, A., Engler, G. and Inzé, D. (2002). Control of proliferation, endoreduplication and differentiation by the Arabidopsis E2Fa/DPa transcription factor. EMBO J. 21: 1360-1368.
del Pozo, J. C., Boniotti, M. B. and Gutierrez, C. (2002). Arabidopsis E2Fc functions in cell division and is degraded by the ubiquitin-SCFAtSKP2 pathway in response to light. Plant Cell 14: 3057-71.
Diffley, J. F. and Labib, K. (2002). The chromosome replication cycle. J. Cell. Sci. 115: 869-872.
Dimova, D. K. and Dyson, N. J. (2005). The E2F transcriptional network: old acquaintances with new faces. Oncogene 24: 2810-2826.
Doucet-Chabeaud, G., Godon, C., Brutesco, C., de Murcia, G. and Kazmaier, M. (2001). Ionising radiation induces the expression of PARP-1 and PARP-2 genes in Arabidopsis. Mol. Genet. Genomics 265: 954-963.
Erickson, R. O. (1976). Modeling of plant growth. Annu. Rev. Plant Physiol. 27: 407-434.
Exner, V., Taranto, P., Schonrock, N., Gruissem, W. and Hennig, L. (2006). Chromatin assembly factor CAF-1 is required for cellular differentiation during plant development. Development 133: 4163-4172.
Forsburg, S. L. (2004). Eukaryotic MCM proteins: beyond replication initiation. Microbiol. Mol. Biol. Rev. 68: 109-131.
Gambus, A., Jones, R. C., Sanchez-Diaz, A., Kanemaki, M., van Deursen, F., Edmondson, R. D. and Labib, K. (2006). GINS maintains association of Cdc45 with MCM in replisome progression complexes at eukaryotic DNA replication forks. Nat. Cell. Biol. 8; 358-366.
Giaginis, C., Georgiadou, M., Dimakopoulou, K., Tsourouflis, G., Gatzidou, E., Kourakis, G. and Theocharis, S. (2008). Clinical significance of MCM-2 and MCM-5 expression in colon cancer: association with clinicopathological parameters and tumor proliferation capacity. Dig. Dis. Sci. May 9.
Gietz, D., St Jean, A., Woods, R. A. and Schiestl, R. H. (1992). Improved method for high efficiency transformation of intact yeast cells. Nucleic Acids Res. 20: 1425.
Gillespie, P. J., Li, A. and Blow, J. J. (2001). Reconstitution of licensed replication origins on Xenopus sperm nuclei using purified proteins. BMC Biochem. 2: 15.
Inzé, D. and De Veylder, L. (2006). Cell cycle regulation in plant development. Annu. Rev. Genet. 40: 77-105.

Kamimura, Y., Tak, Y. S., Sugino, A. and Araki, H. (2001). Sld3, which interacts with Cdc45 (Sld4), functions for chromosomal DNA replication in Saccharomyces cerevisiae. EMBO J. 20: 2097-2107.
Kanemaki, M. and Labib, K. (2006). Distinct roles for Sld3 and GINS during establishment and progression of eukaryotic DNA replication forks. EMBO J. 25: 1753-1763
Karimi, M., Inzé, D. and Depicker, A. (2002). GATEWAY™ vectors for Agrobacterium-mediated plant transformation. Trends Plant Sci. 7: 193-195.
Karoui, M., Tresallet, C., Julie, C., Zimmermann, U., Staroz, F., Brams, A., Muti, C., Boulard, C., Robreau, A. M., Puy, H., Malafosse, R., Penna, C., Pruvot, F. R., Thiery, J. P., Boileau, C., Rougier, P., Nordlinger, B., Radvanyi, F., Franc, B., and Hofmann-Radvanyi, H. (2004) Loss of heterozygosity on 10q and mutational status of PTEN and BMPR1A in colorectal primary tumours and metastases. Br J Cancer 90, 1230-1234
Kearsey, S. E. and Labib, K. (1998). MCM proteins: evolution, properties, and role in DNA replication. Biochim. Biophys. Acta 1398: 113-136.
Kilian, J., Whitehead, D., Horak, J., Wanke, D., Weinl, S., Batistic, O., D′ Angelo, C., Bomberg-Bauer, E., Kudla, J., Harter, K. (2007). The AtGenExpress global stress expression data set: protocols, evaluation and model data analysis of UV-B light, drought and cold stress response. Plant J. 50: 347-363.
Kosugi, S, and Ohashi, Y. (2002). E2Ls, E2F-like repressors of Arabidopsis that bind to E2F sites in a monomeric form. J. Biol. Chem. 277: 16553-16558.
Kulukian A, Han J S, Cleveland D W. (2009) Unattached kinetochores catalyze production of an anaphase inhibitor that requires a Mad2 template to prime Cdc20 for BubR1 binding. Dev Cell. 16(1):105-17.
Labib, K. and Diffley, J. F. (2001). Is the MCM2-7 complex the eukaryotic DNA replication fork helicase? Curr. Opin. Genet. Dev. 11: 64-70.
Labib, K., Tercero, J. A. and Diffley, J. F. (2000). Uninterrupted MCM2-7 function required for DNA replication fork progression. Science 288: 1643-1647.
Lemmens, I., Lievens, S., Eyckerman, S and Tavernier, J. (2006). Reverse MAPPIT detects disruptors of protein-protein interactions in human cells. Nat. Protoc. 1: 92-97.

Maere, S., Heymans, K., Kuiper, M. (2005). BiNGO: a Cytoscape plugin to assess overrepresentation of gene ontology catergories in biological networks. Bioinformatics 21:3448-3449.

Malureanu L A, Jeganathan K B, Hamada M, Wasilewski L, Davenport J, van Deursen J M. (2009) BubR1 N terminus acts as a soluble inhibitor of cyclin B degradation by APC/C(Cdc20) in interphase. Dev Cell. 16(1):118-31.

Masumoto, H., Muramatsu, S., Kamimura, Y. and Araki, H. (2002). S-Cdk-dependent phosphorylation of Sld2 essential for chromosomal DNA replication in budding yeast. Nature 415: 651-655.
Mihaila, D., Gutierrez, J. A., Rosenblum, M. L., Newsham, I. F., Bogler, O., and Rempel, S. A. (2003) Meningiomas: analysis of loss of heterozygosity on chromosome 10 in tumor progression and the delineation of four regions of chromosomal deletion in common with other cancers. Clin Cancer Res 9, 4435-4442
Molinier, J., Oakeley, E. J., Niederhauser, O., Kovalchuk, I., Hohn, B. (2005). Dynamic response of plant genome to ultraviolet radiation and other genotoxic stresses. Mutat. Res. 571:235-47.
Obayashi, T., Kinoshita, K., Nakai, K., Shibaoka, M., Hayashi, S., Saeki, M., Shibata, D., Saito, K. and Ohta, H. (2007). ATTED-II: a database of co-expressed genes and cis elements for identifying co-regulated gene groups in Arabidopsis. Nucleic Acids Res. 35: D863-D869.
Perou, C. M., Sorlie, T., Eisen, M. B., van de Rijn, M., Jeffrey, S. S., Rees, C. A., Pollack, J. R., Ross, D. T., Johnsen, H., Akslen, L. A., et al. (2000). Molecular portraits of human breast tumours. Nature 406, 747-752
Pham, H. M., Arganarez, E. R., Groschel, B., Trono, D and Lama, J. (2004). Lentiviral Vectors Interfering with Virus-Induced CD4 Down-Modulation Potently Block Human Immunodeficiency Virus Type 1 Replication in Primary Lymphocytes. J. Vir. 78: 13072-13071.
Ramirez-Parra, E., Fründt, C. and Gutierrez, C. (2003). A genome-wide identification of E2F regulated genes in Arabidopsis. Plant J. 33: 801-811.
Ramirez-Parra, E., Lopez-Matas, M. A., Fründt, C. and Gutierrez, C. (2004). Role of an atypical E2F transcription factor in the control of Arabidopsis cell growth and differentiation. Plant Cell 16: 2350-2363.
Sakwe, A. M., Nguyen, T., Athanasopoulos, V., Shire, K. and Frappier, L. (2007). Identification and characterization of a novel component of the human minichromosome maintenance complex. Mol. Cel. Biol. 27: 3044-3055.
Schubert, V., Kim, Y. M., Schubert, I. (2008). Arabidopsis sister chromatids often show complete alignment or separation along a 1.2 Mb euchromatic region but no cohesion “hot spots”. Chromosoma 117: 261-266.
Sorlie, T., Perou, C. M., Tibshirani, R., Aas, T., Geisler, S., Johnsen, H., Hastie, T., Eisen, M. B., van de Rijn, M., Jeffrey, S. S., et al. (2001). Gene expression patterns of breast carcinomas distinguish tumor subclasses with clinical implications. Proceedings of the National Academy of Sciences of the United States of America 98, 10869-10874. Sorlie, T., Tibshirani, R., Parker, J., Hastie, T., Marron, J. S., Nobel, A., Deng, S., Johnsen, H., Pesich, R., Geisler, S., et al. (2003). Repeated observation of breast tumor subtypes in independent gene expression data sets. Proceedings of the National Academy of Sciences of the United States of America 100, 8418-8423.
Stuart, J. M., Segal, E., Koller, D. and Kim, S. K. (2003). A gene-coexpression network for global discovery of conserved genetic modules. Science 302: 249-255.
Takayama, Y., Kamimura, Y., Okawa, M., Muramatsu, S., Sugino, A. and Araki, H. (2003). GINS, a novel multiprotein complex required for chromosomal DNA replication in budding yeast. Genes Dev. 17: 1153-1165.
Tavernier, J., Eyckerman, S., Lemmens, I., Van der Heyden, J., Vandekerckhove, J. And Van Ostade, X. (2002). MAPPIT: a cytokine receptor-based two-hybrid method in mammalian cells. Clin. Ecp. Allergy 32: 1397-1404.
Tye, B. K. (1999). MCM proteins in DNA replication. Annu. Rev. Biochem. 68: 649-686.
Tye, B. K. and Sawyer, S. (2000). The hexameric eukaryotic MCM helicase: building symmetry from nonidentical parts. J. Biol. Chem. 275: 34833-34836.
Valvekens, D., Van Montagu, M. and Van Lijsebettens, M. (1988). Agrobacterium tumefaciens-mediated transformation of Arabidopsis thaliana root explants by using kanamycin selection. Proc. Natl. Acad. Sci. USA 85: 5536-5540.
Van Leene, J., Stals, H., Eeckhout, D., Persiau, G., Van De Slijke, E., Van Isterdael, G., De Clercq, A., Bonnet, E., Laukens, K., Remmerie, N., Henderickx, K., De Vijlder, T., Abdelkrim, A., Pharazyn, A., Van Onckelen, H., Inzé, D., Witters, E. and De Jaeger, G. (2007). A Tandem Affinity Purification-based Technology Platform to Study the Cell Cycle Interactome in Arabidopsis thaliana. Mol. Cell. Proteomics 6: 1226-1238.
Vandepoele, K., Vlieghe, K., Florquin, K., Hennig, L., Beemster, G. T. S., Gruissem, W., Van de Peer, Y., Inzé, D. and De Veylder, L. (2005). Genome-wide identification of potential plant E2F target genes. Plant Physiol. 139: 316-328. Vandesompele, J., De Preter, K., Pattyn, F., Poppe, B., Van Roy, N., De Paepe, A., Speleman, F. (2002). Accurate normalization of real-time quantitative RT-PCR data by geometric averaging of multiple internal control genes. Genome Biol. 3: 1-12
Vlieghe, K., Boudolf, V., Beemster, G. T., Maes, S., Magyer, Z., Atanassova, A., de Almeida Engler, J., De Groodt, R., Inzé, D. and De Veylder, L. (2005). The DP-E2F-like gene DEL1 controls the endocycle in Arabidopsis thaliana. Curr. Biol. 15: 59-63.
Walter, M., Chaban, C., Schütze, K., Batistic, O., Weckermann, K., Näke, C., Blazevic, D., Grefen, C., Schumacher, K., Oecking, C., Harter, K. and Kudla, J. (2004). Visualization of protein interactions in living plant cells using bimolecular fluorescence complementation. Plant J. 40: 428-438.
Wei, H., Persson, S., Mehta, T., Srinivasasainagendra, V., Chen, L., Page, G. P., Somerville, C. and Loraine, A. (2006). Transcriptional coordination of the metabolic network in Arabidopsis. Plant Physiol. 142: 762-774.
Wiznerowicz, M. and Didier, T. (2005). Harnessing HIV for therapy, basic research and biotechnology. TRENDS in Biotechnology. 23: 42-47. Zou, L. and Stillman, B. (2000). Assembly of a complex containing Cdc45p, replication protein A, and Mcm2p at replication origins controlled by S-phase cyclin-dependent kinases and Cdc7p-Dbf4p kinase. Mol. Cell. Biol. 20: 3086-3096.
Zuffery, R., Nagy, D., Mandel, R. J., Naldini, L. and Trono, D. (1997). Multiply attenuated lentiviral vector achieves efficient delivery in vivo. Nat. Biotechnol. 15: 871-875.

1.-5. (canceled)

6. A method of diagnosing cancer in a subject and/or coming to a prognosis as to cancer in a subject suffering therefrom, wherein the improvement comprises:
utilizing ETG1 or an ETG1 ortholog for the diagnosis and/or prognosis of cancer in the subject.

7. The method according to claim 6, wherein an ETG1 ortholog is utilized.

8. The method according to claim 7, wherein the ETG1 ortholog is human ortholog C10orf119.

9. A method of treating a cancer in a subject, wherein the improvement comprises:
utilizing an ETG1 ortholog to treat the cancer.

10. The method according to claim 9, wherein the ETG1 ortholog is the human ortholog C10orf119.

11. A method of screening compounds for biological activity, wherein the improvement comprises:
utilizing ETG1 or an ETG1 ortholog to screen compounds for compounds that interfere with the interaction of ETG1 or an ETG1 ortholog with the minichromosome maintenance complex (“MCM”).

12. A method of treating cancer in a subject, the method comprising:
interfering with ETG1 or an ETG1 ortholog's interaction with the minichromosome maintenance complex (“MCM”)
so as to treat the cancer in the subject.

13. The method according to claim 12, wherein interfering with the interaction of ETG1 or an ETG1 ortholog with the MCM comprises administering to the subject a compound that interferes with the interaction of ETG1 or an ETG1 ortholog with the MCM.
